(19) United States
(12) Patent Application Publication
(10) Pub. No.: US 2010/0281575 A1
(43) Pub. Date: Nov. 4, 2010

(54) ROSE CONTAINING FLAVONE AND MALVIDIN, AND METHOD FOR PRODUCTION THEREOF

(75) Inventors: Yoshikazu Tanaka, Osaka (JP); Yukihisa Katsumoto, Osaka (JP); Yuko Fukui, Osaka (JP); Masako Mizutani, Osaka (JP); Noriko Nakamura, Osaka (JP); Junichi Togami, Osaka (JP)

Correspondence Address: DRINKER BIDDLE & REATH (DC), 1500 K STREET, N.W., SUITE 1100, WASHINGTON, DC, 20005-1209, US

(73) Assignee: International Flower Developments Proprietary Limi ted, Bundoora (AU)

(21) Appl. No.: 12/664,998

(22) PCT Filed: Jun. 19, 2008
(86) PCT No.: PCT/JP2008/061593

§ 371 Date: Dec. 16, 2009

(30) Foreign Application Priority Data
Jun. 20, 2007 (JP) 2007-162959

(51) Int. Cl. A01H 1/00; A01H 5/00

(52) U.S. Cl. 800/282; 800/323

(57) ABSTRACT

The invention provides a rose characterized by comprising a flavone and malvidin added by a genetic modification method. The flavone and malvidin are typically produced by expression of a transferred flavone synthase gene, pansy flavonoid 3′,5′-hydroxylase gene and anthocyanin methyltransferase gene. The flavone synthase gene is, for example a flavone synthase gene of the family Scrophulariaceae, and specifically it may be the flavone synthase gene of snapdragon of the family Scrophulariaceae, or the flavone synthase gene of torenia of the family Scrophulariaceae. The flavonoid 3′,5′-hydroxylase gene is, for example, the pansy flavonoid 3′,5′-hydroxylase gene. The anthocyanin methyltransferase gene is, for example, the methyltransferase gene of torenia of the family Scrophulariaceae.

### TECHNICAL FIELD

The present invention relates to an artificially made rose containing a flavone and malvidin. The invention further relates to a method for modifying rose petal color by a co-pigmentation effect which is produced by adding a flavone and malvidin by genetic engineering, and particularly to a method for altering petal color toward blue.

### BACKGROUND ART

Flowers are plant reproductive organs that are required for production of seeds for subsequent generations. Formation of seeds requires adhesion of pollen onto the pistils, and fertilization. Pollen is usually carried by insects such as bees and butterflies, by hummingbirds, and rarely by bats. The role of flower petals is to attract these organisms that carry pollen, and plants have developed modifications to flower color, shape and coloring pattern for this purpose.

Since flower color is also the most important trait for ornamental flowers, flowers of various colors have traditionally been bred by cross-breeding. However, it is rare for one plant variety to have different flower colors, and for example, crossbreeding has not produced any purple to blue varieties for rose (Rosa hybrida), carnation (Dianthus caryophyllus), chrysanthemum (Chrysanthemum morifolium) or lily (Lilium spp.), or bright red varieties for Japanese garden iris (Iris ensata Thumb.) or gentian (Gentiana triflora).

Light yellow to red or blue flower colors are generally due to the presence of flavonoids and anthocyanins (colored glucosides belonging to the flavonoid class). Flavonoids are common secondary metabolites of plants, and they have a basic C₆C₃C₆ backbone and are synthesized from phenylalanine and malonyl-CoA, as shown below. They are classified as flavones, flavonols, etc. according to the oxidation states of the C-rings.

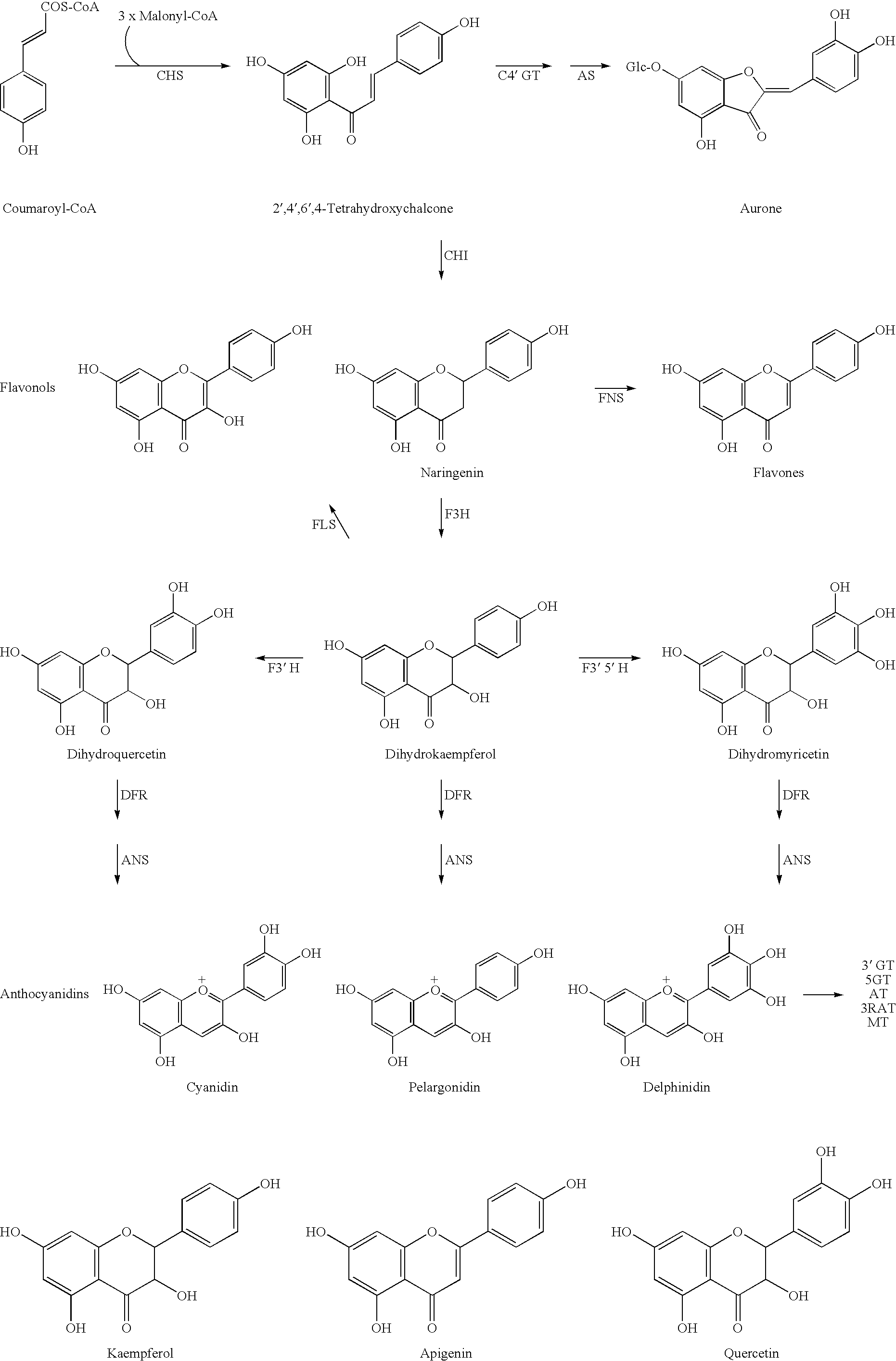

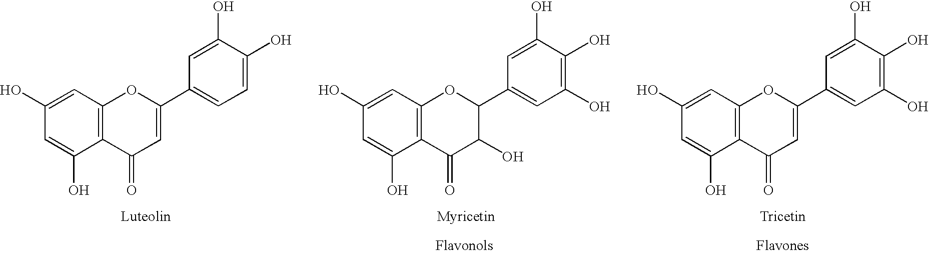

Flavonoids absorb ultraviolet rays and remove radicals, and their original function is therefore believed to be protection of plant bodies from various forms of stress. They have also received attention in recent years as healthy components (see Harborne and Williams 2000 Phytochemistry 55, 481-504).

Several hundred molecular species of colored anthocyanins are known, and of the chromophoric anthocyanidins, the most common are the following 6 types: (1) pelargonidin abundant in orange to red flowers, (2) cyanidin and peonidin abundant in red to crimson flowers, and (3) delphinidin, petunidin and malvidin abundant in violet to blue flowers.

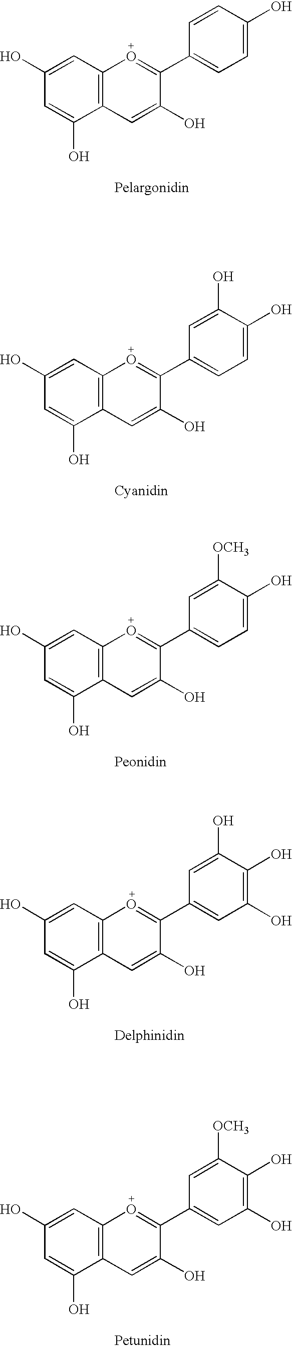

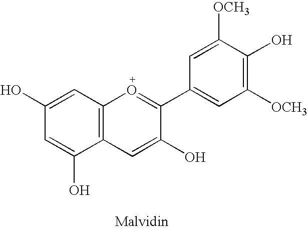

The anthocyanin structure has a major effect on color. An increased number of hydroxyl groups on the B ring of the anthocyanin results in a greater degree of blue. Delphinidin-type anthocyanins are bluer than pelargonidin-type anthocyanins and cyanidin-type anthocyanins. Biosynthesis of flavonoids including anthocyanins is highly conserved across plant species. Flavonoids are biosynthesized in the cytosol, and after addition of sugars and acyl groups, they are transported to the vacuoles and accumulated (see Tanaka et al. 2005 Plant Cell, Tissue and Organ Culture 80, 1-24 and Tanaka and Brugliera 2006 Ainsworth, ed. Flowering and its manipulation, pp. 201-239, Blackwell Publishing Ltd.).

The structural genes of the enzymes involved in the biosynthesis have all been cloned. Creating recombinant plants therefore allows modification of the structures and amounts of flavonoids that are accumulated in flowers by artificial expression of their genes, thereby altering the flower color (Tanaka et al. 2005 Plant Cell, Tissue and Organ Culture 80, 1-24, Tanaka and Brugliera 2006 Ainsworth, Flowering and its manipulation, pp. 201-239, Blackwell Publishing Ltd.). For example, for carnations or roses that cannot produce delphinidin in the petals, the flavonoid 3′,5′-hydroxylase (hereinafter abbreviated as “F3′5′H”) gene necessary for synthesis of delphinidin has been expressed to produce delphinidin to create an artificial blue flower (see Tanaka 2006 Phytochemistry Reviews 5, 283-291).

Such methods of artificially modifying plant metabolism are sometimes called “metabolic engineering”. In order to modify metabolism for accumulation of a substance of interest expression of the gene of the enzyme that produces the substance of interest in a recombinant plant is possible, but in many cases competition with endogenous enzymes of the same plant results in little or absolutely no accumulation of the substance of interest, and therefore no industrially useful trait is obtained.

For example, petunias (Petunia hybrida) do not accumulate pelargonidin due to the specificity of dihydroflavonol reductase (hereinafter abbreviated as “DFR”), and therefore no natural varieties exist with orange-colored flowers.

While orange petunias that accumulate pelargonidin by transfer of the DFR gene from roses or the like have been reported, the accumulation of pelargonidin requires the use of petunia varieties lacking the genes for flavonoid 3′-hydroxylase (hereinafter abbreviated as “F3′H”), F3′5′H and flavonol synthase (hereinafter abbreviated as “FLS”) that compete with DFR, because no change in phenotype is observed when the rose DFR gene is transferred into petunias that do not lack these genes (see Tanaka and Brugliera 2006 Ainsworth, Flowering and its manipulation, pp. 201-239, Blackwell Publishing Ltd.). Consequently, it cannot be predicted whether a compound of interest will be accumulated to exhibit the desired phenotype simply by transferring a gene of interest.

In addition, metabolic engineering often produces unpredictable results. For example, when expression of the flavone synthase gene was inhibited in torenia (Torenia hybrida), the flavone content was reduced and accumulation of flavanones was observed. Accumulation of flavanones would be expected to result in an increased anthocyanin content, but in actuality the anthocyanin content decreased (Ueyama et al. Plant Science, 163, 253-263, 2002). It is therefore difficult to predict changes in metabolites, and persistent modifications have been necessary to obtain desired phenotypes.

Anthocyanins bound with higher numbers of aromatic acyl groups also appear bluer due to an intramolecular copigment effect. Anthocyanins with two or more aromatic acyl groups are known as polyacylated anthocyanins, and they exhibit a stable blue color (see Harborne and Williams 2000 Phytochemistry 55, 481-504).

The color of a flower changes not only by the structure of the anthocyanin pigments themselves as the essential pigments, but also due to copresent flavonoids (also known as copigments), metal ions, and the pH of the vacuoles. For example, flavones or flavonols are typical copigments that form sandwich-like stacking with anthocyanins and render the anthocyanins bluing and deepening color effects (see Goto (1987) Prog. Chem. Org. Natl. Prod. 52). Flavones can thus be considered colorless copigment components. For example, isovitexin, a type of flavone, exhibits a copigment effect for anthocyanins in Japanese garden iris (Iris ensata Thunb.). Isovitexin also stabilizes anthocyanins, thus producing a stabilizing effect on Japanese garden iris flower color (see Yabuya et al. Euphytica 2000 115, 1-5).

Flavones usually exhibit stronger copigment effects than flavonols. For example, analysis of genetically modified carnations has indicated a stronger copigment effect for flavones than flavonols (see Fukui et al. Phytochemistry, 63, 15-23, 2003). Accumulation of flavones is therefore important for creating blue flower color. However, not all plants can produce flavones, and it is known that roses and petunias do not accumulate flavones. In addition to flower color, it is known that flavones play a role in absorption of ultraviolet rays, countering various types of stress, and interaction with microorganisms, and that plants with new traits can be obtained through synthesis of flavones (as a patent document relating to a gene coding for flavone synthase, see Japanese Unexamined Patent Publication No. 2000-279182). However, as yet no examples of flavone-expressing roses have been known.

Flavones are synthesized from flavanones by reaction catalyzed by flavone synthase. Specifically, apigenin is synthesized from naringenin, luteolin is synthesized from eriodictyol and tricetin is synthesized from pentahydroxyflavanone. Flavone synthase exists in two forms, flavone synthase I and flavone synthase II. Both catalyze the same reaction, but are different types of enzymes. Flavone synthase I is a 2-oxoglutaric acid-dependent dioxygenase (see Britsch et al. (1981) Z. Naturforsch 36c pp. 742-750 and Britsch (1990) Arch. Biochem. Biophys. 282 pp. 152-160), while flavone synthase II is a cytochrome P450-type monooxygenase. The structural gene of flavone synthase II can be obtained from torenia, snapdragon, perilla (Perilla frutescens), gerbera (Gerbera hybrida) and gentian (see Tanaka and Brugliera 2006 Ainsworth, Flowering and its manipulation, pp. 201-239, Blackwell Publishing Ltd.).

Flavone synthesis is predictable when the flavone synthase gene is expressed in genetically modified plants that do not produce flavones. However, when the torenia flavone synthase gene is expressed in petunias, it has been reported that the deep violet color of the flower becomes faint (Tsuda et al. Plant Biotechnology, 21, 377-386, 2004). It has also been reported that expression of the gentian-derived flavone synthase gene in tobacco results in flavone synthesis but, likewise, results in a fainter flower color (Nakatsuka et al. 2006, Molecular Breeding 17:91-99). Thus, blue flower color is not always obtained even when flavones are synthesized. The reason for the lack of copigment effect could be an unsuitable ratio of the anthocyanin and flavone contents or unsuitable modification of the anthocyanins and flavones with sugars and acyl groups. These results suggest that it is not possible to increase the blueness of flower color simply by expressing the flavone synthase gene and accumulating flavones.

Roses are the most popular of flowering plants, and they have been cultivated since ancient times. Artificially modified varieties have also been produced in the past several hundred years. Roses have therefore been obtained containing flavonoids such as pelargonidin, cyanidin and flavonols. In recent years as well, roses have been created by genetic modification techniques to produce delphinidin that is not naturally found in roses. However, no flavone-accumulating roses have yet been obtained, either by cross-breeding or by genetic modification. In addition, no roses have yet been obtained that accumulate both a flavone and malvidin.

### DISCLOSURE OF THE INVENTION

The major advantage of using genetic modification for breeding of plants is that, unlike cross-breeding, it allows modifications to plants that cannot be achieved by cross-breeding, and modifications using genes from other organisms. That is, genetic modification allows any gene of an organism of a different species to be transferred into a plant such as a rose, to impart a new ability to the plant. However, unlike model plants such as Arabidopsis (Arabidopsis thaliana) and tobacco (Nicotiana tabacum L.), the functioning of transferred genes in roses is largely dependent on the source of the gene and the promoter used.

According to WO2005/017147, transfer of the flavonoid 3′,5′-hydroxylase gene (F3′5′H) into roses resulted in no expression in the genetically modified rose and no detection of delphinidin when the gene was derived from petunia or gentian, but interestingly, when the gene was derived from pansy it was expressed and imparted to roses the new ability to produce delphinidin. In roses, therefore, it cannot be easily inferred which genes derived from which plant varieties will function when transferred.

When a gene is transferred into chrysanthemums as well, it is difficult to predict whether the gene will function in the chrysanthemums, and it is known that transferred genes lose their function as recombinant chrysanthemums age. The 35S promoter of cauliflower mosaic virus, which is often used for transfer of foreign genes in recombinant plants, has been reported to function in gentian (see Mishiba et al. Plant Journal 2005, 44:541-556).

While it can be assumed that synthesis of flavones in roses can be easily achieved by expressing the flavone synthase gene, it is not easy to predict whether to express the dioxygenase-type or the cytochrome P450-type flavone synthase, or which plant source should be used for the flavone synthase gene, and therefore trial and error is necessary. The copigment effect is a phenomenon produced when anthocyanins and flavones or flavonols are copresent in a certain concentration in the vacuoles, and it has been demonstrated that this requires the flavone or flavonol copigments to undergo glycosylation or other modification more adapted to the condition of glycosylation or other modification of the anthocyanin color sources (see Nature. 2005 Aug. 11; 436(7052):791 and Nature, 358, 515-518 (1992)).

For expression of the necessary color tone it is necessary for the anthocyanins and flavones/flavonols to be in the optimal structural combination, and this requires trial and error in regard to what sort of modifications should exist in the copresent anthocyanins and flavones/flavonols. In addition, because flavanones such as naringenin are rapidly hydroxylated by flavanone 3-hydroxylase (hereinafter abbreviated as “F3H”) in natural roses, flavones are not necessarily synthesized from flavanones even if flavone synthase is functioning in the rose.

It is therefore an object of the present invention to provide roses comprising appropriate pigments for expression of desired color tones in the roses.

As a result of much research directed toward solving the problems mentioned above, the present inventors have completed this invention upon finding that desired color tone expression can be accomplished by artificially adding flavones and malvidin to roses.

Specifically, the present invention provides the following:

1. A rose characterized by comprising a flavone and malvidin added by a genetic modification method.

2. A rose according to 1 above, which comprises a flavone and malvidin by expression of pansy (Viola x wittrockiana) flavonoid 3′,5′-hydroxylase and anthocyanin methyltransferase.

3. A rose according to 1 or 2 above, which comprises malvidin, a flavone and delphinidin by expression of an anthocyanin methyltransferase gene, a flavone synthase gene and the pansy (Viola x wittrockiana) flavonoid 3′,5′-hydroxylase gene.

4. A rose according to any one of 1 to 3 above, wherein the flavone synthase gene is a flavone synthase gene derived from the family Scrophulariaceae.

5. A rose according to 4 above, wherein the flavone synthase gene derived from the family Scrophulariaceae is a flavone synthase gene derived from snapdragon of the family Scrophulariaceae (Scrophulariaceae, Antirrhinum majus).

6. A rose according to 4 above, wherein the flavone synthase gene derived from the family Scrophulariaceae is a flavone synthase gene derived from torenia of the family Scrophulariaceae (Scrophulariaceae, Torenia hybrida).

7. A rose according to 5 above, wherein the flavone synthase gene derived from snapdragon of the family Scrophulariaceae is a gene coding for:

(1) flavone synthase having the amino acid sequence listed as SEQ ID NO: 2,
(2) flavone synthase having the amino acid sequence listed as SEQ ID NO: 2 modified by an addition or deletion of one or several amino acids and/or substitution of one or several amino acids by other amino acids,
(3) flavone synthase having an amino acid sequence with at least 90% sequence identity to the amino acid sequence listed as SEQ ID NO: 2, or
(4) flavone synthase encoded by nucleic acid that hybridizes with nucleic acid having the nucleotide sequence of SEQ ID NO: 1 under highly stringent conditions.

8. A rose according to 6 above, wherein the flavone synthase gene derived from torenia of the family Scrophulariaceae is a gene coding for:

(1) flavone synthase having the amino acid sequence listed as SEQ ID NO: 4,
(2) flavone synthase having the amino acid sequence listed as SEQ ID NO: 4 modified by an addition or deletion of one or several amino acids and/or substitution of one or several amino acids by other amino acids,
(3) flavone synthase having an amino acid sequence with at least 90% sequence identity to the amino acid sequence listed as SEQ ID NO: 4, or
(4) flavone synthase encoded by nucleic acid that hybridizes with nucleic acid having the nucleotide sequence of SEQ ID NO: 3 under highly stringent conditions.

9. A rose according to any one of 2 to 8 above, wherein the pansy flavonoid 3′,5′-hydroxylase gene is a gene coding for:

(1) flavonoid 3′,5′-hydroxylase having the amino acid sequence listed as SEQ ID NO: 8,
(2) flavonoid 3′,5′-hydroxylase having the amino acid sequence listed as SEQ ID NO: 8 modified by an addition or deletion of one or several amino acids and/or substitution of one or several amino acids by other amino acids,
(3) flavonoid 3′,5′-hydroxylase having an amino acid sequence with at least 90% sequence identity to the amino acid sequence listed as SEQ ID NO: 8, or
(4) flavonoid 3′,5′-hydroxylase encoded by nucleic acid that hybridizes with nucleic acid having the nucleotide sequence of SEQ ID NO: 7 under highly stringent conditions.

10. A rose according to any one of 2 to 9, wherein the anthocyanin methyltransferase gene is a gene coding for:

(1) methyltransferase having the amino acid sequence listed as SEQ ID NO: 10,
(2) methyltransferase having the amino acid sequence listed as SEQ ID NO: 10 modified by an addition or deletion of one or several amino acids and/or substitution of one or several amino acids by other amino acids,
(3) methyltransferase having an amino acid sequence with at least 90% sequence identity to the amino acid sequence listed as SEQ ID NO: 10, or
(4) methyltransferase encoded by nucleic acid that hybridizes with nucleic acid having the nucleotide sequence of SEQ ID NO: 9 under highly stringent conditions.

11. A rose according to any one of 2 to 10 above, wherein the flower color is changed with respect to the host before transfer of the anthocyanin methyltransferase gene, flavone synthase gene and pansy 3′,5′-hydroxylase gene.

12. A rose according to 11 above, wherein the change in flower color is a change toward blue.

13. A rose according to 11 or 12 above, wherein the change in flower color is a change such that the hue angle (θ) according to the L*a*b color system chromaticity diagram approaches 270° which is the blue axis.

14. A rose according to 11 above, wherein the change in flower color is a change such that the minimum value of the reflection spectrum of the petal shifts toward the longer wavelength end.

15. A rose portion, descendant, tissue, vegetative body or cell having the same properties as a rose according to any one of 1 to 14 above.

16. A method for modifying the flower color of a rose by a co-pigmentation effect produced by adding a flavone and malvidin by a genetic modification technique.

17. The method according to 16 above, wherein the co-pigmentation effect is an effect of changing the flower color toward blue.

### BEST MODE FOR CARRYING OUT THE INVENTION

### Definition of Terms

The term “rose”, as used throughout the present specification, is a general name for an ornamental plant which is a deciduous shrub of the order Rosales, family Rosaceae, genus Rosa, and it is not limited to any specific variety and includes the entire plant or a portion thereof usually containing the flower.

A reference to a “portion, descendant, tissue, vegetative body or cell” of a “rose”, as used throughout the present specification, means any thing derived from a “rose” so long as it retains the desired genetic trait of a “rose” according to the invention, and it is not limited to any particular entity.

The phrase “highly stringent conditions”, as used throughout the present specification means, for example, conditions of heating the antisense strand and the target nucleic acid overnight at 55° C. in a solution comprising 6×SSC (1×SSC composition: 0.15 M NaCl, 0.015 M sodium citrate, pH 7.0), 0.5% SDS, 5×Denhardt, 100 μg/ml denatured fragmented salmon sperm DNA and 50% formamide, and rinsing under conditions of 0.1×SSC and/or conditions of 60° C. or above, and specifically it refers to any conditions under which the nonspecific signal of the background is essentially absent.

The phrase “hue angle (θ) according to the L*a*b color system chromaticity diagram”, as used throughout the present specification, refers to the hue angle (θ) standardized by the 1976 Commission internationale de l′eclairage (CIE) and adopted in Japan as JIS8729, where 0° is the red direction, 90° is the yellow direction, 180° is the green direction and 270° is the blue direction. Flower color can be represented by a combination of this hue angle and RHS (Royal Horticultural Society) color chart data.

Transfer of flavone synthesis gene, flavonoid 3′,5′-hydroxylase gene and anthocyanin methyltransferase gene

The gene for flavone synthase II derived from perilla was transferred into rose by a known procedure, together with the pansy F3′S′H gene. As a result, no flavones were detected in roses into which the perilla flavone synthase II gene had been transferred, indicating that the gene does not function in rose. On the other hand, flavone was detected in roses into which torenia or snapdragon flavone synthase II genes had been transferred, indicating that the flavone synthesis genes do function in rose.

Flavone-accumulating roses not found in the prior art were thus created. The flavone content (%) of the total flavonoids may be 1% or greater, preferably 5% or greater, more preferably 10% or greater and most preferably 30% or greater. Roses accumulating both anthocyanins and flavones have relatively bluer colors compared to roses containing only the same anthocyanins, thus suggesting that flavone accumulation contributes to the new trait of blueness.

In addition, it was found that when an anthocyanin with a methylated B ring (anthocyanins including malvidin) and a flavone are copresent, a higher copigment effect is exhibited than when a delphinidin-containing anthocyanin and a flavone are copresent, and that by transferring a methyltransferase gene for the B ring in addition to the flavone synthase II gene and pansy F3′5′H gene, it is possible to accumulate methylated delphinidin-type anthocyanins and flavones in rose petals, thus resulting in a bluer color of the rose petals.

By a hybridization test it was also found that the trait of accumulating both delphinidin-type anthocyanins and flavones is transmitted to progeny.

These findings indicate that in plants that do not accumulate flavones or do not accumulate methylated anthocyanins such as malvidins in the petals, a bluer color shade of the petals can be produced by causing these compounds to be accumulated simultaneously. For this purpose it is preferable for the host to be a plant that normally does not accumulate flavones or malvidin, such as a rose.

The enzymes associated with the invention are typically enzymes having specific amino acid sequences listed in the Sequence Listing. However, it is well known that desired enzyme activity can be maintained not only with the natural amino acid sequence of an enzyme, but also with the same amino acid sequence having modifications in regions other than the regions associated with the enzyme activity. Consequently, the enzymes of the invention include proteins having the amino acid sequences specified by the SEQ ID NOs which are modified by an addition or deletion of one or several amino acids and/or by substitution of one or several amino acids with other amino acids, and still maintaining the original enzyme activities, and also proteins having amino acid sequences with at least 90% sequence identity to the specific amino acid sequences specified by the SEQ ID NOs, and maintaining the original enzyme activities.

It is known that for any gene coding for a certain enzyme, there is a high probability that nucleic acid that hybridizes with the gene under highly stringent conditions will code for an enzyme having the same activity as that enzyme. Thus, enzymes encoded by nucleic acids that hybridize with nucleic acids having the nucleotide sequences specified by the SEQ ID NOs under highly stringent conditions, and having the desired enzyme activities, are also included as enzymes according to the invention.

The following genes may therefore be mentioned as enzyme genes within the scope of the invention.
(A) Snapdragon (Antirrhinum majus) flavone synthase gene A gene coding for:
   (1) flavone synthase having the amino acid sequence listed as SEQ ID NO: 2,
   (2) flavone synthase having the amino acid sequence listed as SEQ ID NO: 2 modified by an addition or deletion of one or several amino acids and/or substitution of one or several amino acids by other amino acids,
   (3) flavone synthase having an amino acid sequence with at least 90% sequence identity to the amino acid sequence listed as SEQ ID NO: 2, or
   (4) flavone synthase encoded by nucleic acid that hybridizes with nucleic acid having the nucleotide sequence of SEQ ID NO: 1 under highly stringent conditions.
(B) Torenia (Torenia hybrida) flavone synthase gene A gene coding for:
   (1) flavone synthase having the amino acid sequence listed as SEQ ID NO: 4,
   (2) flavone synthase having the amino acid sequence listed as SEQ ID NO: 4 modified by an addition or deletion of one or several amino acids and/or substitution of one or several amino acids by other amino acids,
   (3) flavone synthase having an amino acid sequence with at least 90% sequence identity to the amino acid sequence listed as SEQ ID NO: 4, or
   (4) flavone synthase encoded by nucleic acid that hybridizes with nucleic acid having the nucleotide sequence of SEQ ID NO: 3 under highly stringent conditions.
(C) Perilla (Perilla frutescens) flavone synthase gene A gene coding for:
   (1) flavone synthase having the amino acid sequence listed as SEQ ID NO: 6,
   (2) flavone synthase having the amino acid sequence listed as SEQ ID NO: 6 modified by an addition or deletion of one or several amino acids and/or substitution of one or several amino acids by other amino acids,
   (3) flavone synthase having an amino acid sequence with at least 90% sequence identity to the amino acid sequence listed as SEQ ID NO: 6, or
   (4) flavone synthase encoded by nucleic acid that hybridizes with nucleic acid having the nucleotide sequence of SEQ ID NO: 5 under highly stringent conditions.
(D) Pansy (Viola x wittrockiana) 3′,5′-hydroxylase gene A gene coding for:
   (1) 3′,5′-hydroxylase having the amino acid sequence listed as SEQ ID NO: 8,
   (2) 3′,5′-hydroxylase having the amino acid sequence listed as SEQ ID NO: 8 modified by an addition or deletion of one or several amino acids and/or substitution of one or several amino acids by other amino acids,
   (3) 3′,5′-hydroxylase having an amino acid sequence with at least 90% sequence identity to the amino acid sequence listed as SEQ ID NO: 8, or
   (4) 3′,5′-hydroxylase encoded by nucleic acid that hybridizes with nucleic acid having the nucleotide sequence of SEQ ID NO: 7 under highly stringent conditions.
(E) Torenia (Torenia hybrida) methyltransferase gene A gene coding for:
   (1) methyltransferase having the amino acid sequence listed as SEQ ID NO: 10,
   (2) methyltransferase having the amino acid sequence listed as SEQ ID NO: 10 modified by an addition or deletion of one or several amino acids and/or substitution of one or several amino acids by other amino acids,
   (3) methyltransferase having an amino acid sequence with at least 90% sequence identity to the amino acid sequence listed as SEQ ID NO: 10, or
   (4) methyltransferase encoded by nucleic acid that hybridizes with nucleic acid having the nucleotide sequence of SEQ ID NO: 9 under highly stringent conditions.

### EXAMPLES

The present invention will now be explained in greater detail by the following examples. However, these examples are merely for the purpose of illustration of the invention and are not intended to restrict the scope of the invention in any way.

### Example 1

### Simulation of Flavone Copigment Effect with Anthocyanins

Anthocyanins were prepared first for simulation of the flavone copigment effect with anthocyanins. Cyanin was extracted and purified from petals of the rose variety “Rote Rose” (rose cv. “Rote Rose”). Delphin was obtained by alkali hydrolysis of the pigment extracted from petals of the verbena variety “Tapien Violet” (verbena cv. “Tapien Violet” or verbena variety Sunmaref TP-V (“Tapien Violet”)(“Tapien” is a Trade Mark registered in Japan)), followed by purification. Malvin and luteolin 7-O-glucoside were purchased from Funakoshi Corp.

The flavone (luteolin 7-O-glucoside) was added to each anthocyanin prepared in this manner, at 0, 1, 2 and 4 equivalent molar concentrations in a buffering solution at pH 5.0, and the absorption spectra were measured. The anthocyanins used were cyanin (cyanidin 3,5-diglucoside), delphin (delphinidin 3,5-diglucoside) and malvin (malvidin 3,5-diglucoside). The anthocyanin concentrations for cyanin, delphin and malvin were 1 mM.

As shown in Tables 1 and 2, addition of the flavone increased the absorbance of the anthocyanin aqueous solutions and the degree of change (absorbance ratio) was greatest with malvin. The absorption maxima (λmax) were also shifted toward the long wavelength end with addition of the flavone. The degree of change was greatest with malvin, and then with delphin. Upon evaluation of the color shade value based on the L*a*b* color system, addition of the flavone was found to produce a bluer color shade and increased chroma. This effect was most notable with malvin. That is, it was demonstrated that the luteolin 7-O-glucoside copigment effect was exhibited to the greatest extent with malvin.

TABLE 1
Absorption maxima of anthocyanin aqueous
solutions with flavone addition
(λmax: units: nm)

| | Flavone addition | | | |
|---|---|---|---|---|
| Anthocyanin | 0 | 1 equiv | 2 equiv | 4 equiv |
| Cyanin | 522.5 | 540.5 | 546.0 | 545.0 |
| (Cyanidin 3,5-diglucoside) | | | | |
| Delphin | 526.0 | 564.0 | 569.0 | 569.5 |
| (Delphinidin 3,5-diglucoside) | | | | |
| Malvin | 528.5 | 568.5 | 570.5 | 572.5 |
| (Malvidin 3,5-diglucoside) | | | | |

TABLE 2
Absorbance ratios at λmax with respect to no flavone addition

| | Flavone addition | | | |
|---|---|---|---|---|
| Anthocyanin | 0 | 1 equiv | 2 equiv | 4 equiv |
| Cyanin | 1.000 | 2.044 | 2.425 | 2.363 |
| (Cyanidin 3,5-diglucoside) | | | | |
| Delphin | 1.000 | 2.917 | 4.248 | 4.798 |
| (Delphinidin 3,5-diglucoside) | | | | |
| Malvin | 1.000 | 5.194 | 7.775 | 9.219 |
| (Malvidin 3,5-diglucoside) | | | | |

### Example 2 (Reference Example)

### Transfer of Pansy F3′5′H#40 Gene and Perilla Flavone Synthase Gene into Rose Variety “Lavande”

The perilla flavone synthase gene-containing plasmid pYFS3 described in Japanese Unexamined Patent Publication No. 2000-279182 was digested with XbaI and then blunt ended and digested with BamHI to obtain an approximately 1.8 kb perilla flavone synthase gene fragment. Separately, pSPB906 described in WO2005/017147 was digested with XhoI and then blunt ended and further digested with BamHI. The perilla flavone synthase gene fragment was inserted between the flush ends and the BamHI cleavage site to obtain plasmid 906-pYFS3. Plasmid 906-pYES3 comprises the perilla flavone synthase gene between the El₂35S promoter and D8 terminator (both described in WO2005/017147).

A plasmid obtained by inserting a fragment of the pansy F3′5′H#40 gene, cut out from pCGP1961 described in WO2005/017147 by partial digestion with BamHI and XhoI, at the BamHI and SalI sites of pSPB176 reported by Ueyama et al. (Ueyama et al. Plant Science, 163, 253-263, 2002), was designated as pSPB575. At the AscI site of this plasmid there was inserted an approximately 3.4 kb perilla flavone synthase gene expression cassette obtained by digesting the aforementioned plasmid 906-pYFS3 with AscI. Of the obtained plasmids, the vector having the F3′5′H#40 gene expression cassette and the perilla flavone synthase expression cassette linked in the same direction was designated as pSPB1310. This plasmid constitutively expresses the pansy F3′5′H#40 gene and the perilla flavone synthase gene in plants.

Plasmid pSPB1310 constructed in this manner was transferred into the mauve rose variety “Lavande”, and 55 transformants were obtained. Delphinidin accumulation was confirmed in 49 of 50 pigment-analyzed transformants, with a maximum delphinidin content of 70% (average: 26%). However, absolutely no flavones were detected, and it was therefore concluded that the perilla flavone synthase gene does not function in rose cells.

The analysis values for representative transformants are shown in Table 3 below.

TABLE 3

| | | Anthocyanidin | | | Flavonol | | | Flavone | | | |
|---|---|---|---|---|---|---|---|---|---|---|---|
| Plant | Del | (mg/g) | | | (mg/g) | | | (mg/g) | | | |
| No. | (%) | Del | Cya | Pel | M | Q | K | Tri | Lut | Api | Total |
| Control | 0.0 | 0.000 | 0.078 | 0.000 | 0.000 | 0.451 | 0.078 | 0.000 | 0.000 | 0.000 | 0.000 |
| 1 | 70.3 | 0.105 | 0.045 | 0.000 | 0.253 | 0.152 | 0.017 | 0.000 | 0.000 | 0.000 | 0.000 |
| 2 | 67.1 | 0.098 | 0.048 | 0.000 | 0.379 | 0.291 | 0.026 | 0.000 | 0.000 | 0.000 | 0.000 |
| 3 | 50.7 | 0.060 | 0.058 | 0.000 | 0.326 | 0.289 | 0.013 | 0.000 | 0.000 | 0.000 | 0.000 |
| 4 | 60.6 | 0.050 | 0.033 | 0.000 | 0.216 | 0.188 | 0.007 | 0.000 | 0.000 | 0.000 | 0.000 |
| 5 | 66.1 | 0.073 | 0.037 | 0.000 | 0.608 | 0.380 | 0.045 | 0.000 | 0.000 | 0.000 | 0.000 |
| 6 | 67.7 | 0.055 | 0.026 | 0.000 | 0.536 | 0.319 | 0.039 | 0.000 | 0.000 | 0.000 | 0.000 |
| 7 | 56.9 | 0.062 | 0.047 | 0.000 | 0.253 | 0.201 | 0.009 | 0.000 | 0.000 | 0.000 | 0.000 |
| 8 | 52.5 | 0.109 | 0.099 | 0.000 | 0.307 | 0.438 | 0.034 | 0.000 | 0.000 | 0.000 | 0.000 |
| 9 | 50.4 | 0.073 | 0.072 | 0.000 | 0.281 | 0.362 | 0.013 | 0.000 | 0.000 | 0.000 | 0.000 |
| 10 | 61.9 | 0.085 | 0.052 | 0.000 | 0.228 | 0.192 | 0.008 | 0.000 | 0.000 | 0.000 | 0.000 |

Control: Lavande control
Del: Delphinidin,
Cya: Cyanidin,
Pel: Pelargonidin,
M: Myricetin,
Q: Quercetin,
K: Kaempferol,
Tri: Tricetin,
Lut: Luteolin,
Api: Apigenin
Del (%): Proportion of delphinidin in total anthocyanidins

### Example 3

### Transfer of Pansy F3′5′H#40 Gene and Torenia Flavone Synthase Gene into rose Variety “Lavande”

A plasmid obtained by inserting the torenia flavone synthase gene reported by Akashi et al. (Plant Cell Physiol 40, 1182-1186, 1999) at the EcoRI and XhoI sites of plasmid pBluescript II SK(−) was designated as pSPB426. After digestion of this plasmid with KpnI, it was blunt ended and further digested with BamHI to obtain an approximately 1.7 kb torenia flavone synthase gene fragment. Separately, pSPB906 described in WO2005/017147 was digested with XhoI and then blunt ended and further digested with BamHI. The torenia flavone synthase gene fragment was inserted between the blunt ends and the BamHI cleavage site to obtain plasmid 906-426.

A plasmid obtained by inserting a fragment of the pansy F3′5′H#40 gene, cut out from pCGP1961 described in WO2005/017147 by partial digestion with BamHI and XhoI, at the BamHI and SalI sites of pSPB176 reported by Ueyama et al. (Ueyama et al. Plant Science, 163, 253-263, 2002), was designated as pSPB575. At the AscI site of this plasmid there was inserted an approximately 3.3 kb torenia flavone synthase gene expression cassette obtained by digesting the aforementioned plasmid 906-426 with AscI. Of the obtained plasmids, the vector having the F3′5′H#40 gene expression cassette and the torenia flavone synthase expression cassette linked in the same direction was designated as pSPB1309. This plasmid constitutively expresses the pansy F3′5′H#40 gene and the torenia flavone synthase gene in plants.

Plasmid pSPB1309 constructed in this manner was transferred into the mauve rose variety “Lavande”, and 50 transformants were obtained. Delphinidin accumulation was confirmed in 36 of 38 pigment-analyzed transformants, with a maximum delphinidin content of 45% (average: 12%). Also, novel accumulation of flavones (luteolin and apigenin) was confirmed in 35 transformants, due to the action of the torenia flavone synthase gene. At maximum, the total amount of flavones was a high content of 1.68 mg per 1 g of fresh petal weight.

The analysis values for representative transformants are shown in Table 4 below.

TABLE 4

| | | Anthocyanidin | | | Flavonol | | | Flavone | | | |
|---|---|---|---|---|---|---|---|---|---|---|---|
| Plant | Del | (mg/g) | | | (mg/g) | | | (mg/g) | | | |
| No. | (%) | Del | Cya | Pel | M | Q | K | Tri | Lut | Api | Total |
| Control | 0.0 | 0.000 | 0.078 | 0.000 | 0.000 | 0.451 | 0.078 | 0.000 | 0.000 | 0.000 | 0.000 |
| 1 | 10.1 | 0.012 | 0.104 | 0.000 | 0.000 | 0.489 | 0.010 | 0.000 | 0.086 | 0.000 | 0.086 |
| 2 | 9.6 | 0.008 | 0.079 | 0.000 | 0.000 | 0.446 | 0.048 | 0.000 | 0.089 | 0.000 | 0.089 |
| 3 | 10.4 | 0.009 | 0.079 | 0.000 | 0.071 | 0.651 | 0.264 | 0.000 | 0.020 | 0.000 | 0.020 |
| 4 | 44.9 | 0.031 | 0.038 | 0.000 | 0.000 | 0.359 | 0.027 | 0.000 | 1.684 | 0.000 | 1.684 |
| 5 | 33.2 | 0.014 | 0.027 | 0.000 | 0.000 | 0.203 | 0.009 | 0.000 | 1.171 | 0.009 | 1.180 |
| 6 | 37.3 | 0.013 | 0.021 | 0.000 | 0.000 | 0.121 | 0.012 | 0.000 | 0.997 | 0.007 | 1.003 |
| 7 | 39.0 | 0.013 | 0.021 | 0.000 | 0.000 | 0.000 | 0.029 | 0.000 | 1.153 | 0.008 | 1.161 |
| 8 | 35.8 | 0.024 | 0.043 | 0.000 | 0.000 | 0.205 | 0.000 | 0.000 | 1.642 | 0.010 | 1.652 |
| 9 | 36.1 | 0.013 | 0.024 | 0.000 | 0.000 | 1.223 | 0.006 | 0.000 | 0.785 | 0.000 | 0.785 |
| 10 | 32.2 | 0.010 | 0.020 | 0.000 | 0.000 | 0.171 | 0.027 | 0.000 | 0.917 | 0.007 | 0.924 |

Control: Lavande control
Del: Delphinidin,
Cya: Cyanidin,
Pel: Pelargonidin,
M: Myricetin,
Q: Quercetin,
K: Kaempferol,
Tri: Tricetin,
Lut: Luteolin,
Api: Apigenin
Del (%): Proportion of delphinidin in total anthocyanidins

### Example 4

### Transfer of Pansy F3′5′H#40 Gene and Torenia Flavone Synthase Gene into Rose Variety “WKS124”

Plasmid pSPB1309 described in Example 3 was transferred into the salmon-pink rose variety “WKS124”, and 40 transformants were obtained. Delphinidin accumulation was confirmed in 26 of 27 pigment-analyzed transformants, with a maximum delphinidin content of 96% (average: 81%). Also, novel accumulation of flavones (tricetin, luteolin and apigenin) was confirmed in 26 transformants, due to the action of the torenia flavone synthase gene. At maximum, the total amount of flavones was a high content of 4.41 mg per 1 g of fresh petal weight.

The analysis values for representative transformants are shown in Table 5 below.

TABLE 5

| | | Anthocyanidin | | | Flavonol | | | Flavone | | | |
|---|---|---|---|---|---|---|---|---|---|---|---|
| Plant | Del | (mg/g) | | | (mg/g) | | | (mg/g) | | | |
| No. | (%) | Del | Cya | Pel | M | Q | K | Tri | Lut | Api | Total |
| Control | 0.0 | 0.000 | 0.006 | 0.073 | 0.000 | 0.076 | 3.312 | 0.000 | 0.000 | 0.000 | 0.000 |
| 1 | 84.8 | 0.326 | 0.045 | 0.014 | 0.427 | 0.026 | 0.797 | 0.941 | 0.122 | 0.394 | 1.456 |
| 2 | 86.6 | 0.567 | 0.084 | 0.003 | 0.806 | 0.096 | 0.218 | 2.148 | 1.863 | 0.395 | 4.406 |
| 3 | 82.4 | 0.191 | 0.029 | 0.011 | 0.000 | 0.139 | 0.626 | 1.095 | 0.055 | 0.838 | 1.988 |
| 4 | 83.4 | 0.448 | 0.083 | 0.007 | 0.000 | 0.037 | 0.434 | 1.157 | 0.131 | 0.486 | 1.774 |
| 5 | 80.1 | 0.340 | 0.072 | 0.012 | 0.185 | 0.064 | 0.735 | 0.872 | 0.111 | 0.401 | 1.384 |
| 6 | 83.5 | 0.362 | 0.065 | 0.007 | 0.000 | 0.090 | 0.676 | 1.642 | 0.229 | 0.777 | 2.647 |
| 7 | 88.5 | 0.895 | 0.111 | 0.006 | 0.000 | 0.095 | 0.288 | 1.501 | 0.113 | 0.046 | 1.660 |
| 8 | 87.3 | 0.862 | 0.123 | 0.003 | 0.275 | 0.092 | 0.200 | 1.286 | 0.127 | 0.082 | 1.495 |
| 9 | 89.6 | 0.252 | 0.029 | 0.001 | 0.126 | 0.049 | 0.097 | 2.558 | 0.332 | 0.295 | 3.184 |
| 10 | 81.3 | 0.101 | 0.022 | 0.001 | 0.065 | 0.031 | 0.146 | 1.822 | 0.215 | 0.405 | 2.442 |

Control: WKS124 control
Del: Delphinidin,
Cya: Cyanidin,
Pel: Pelargonidin,
M: Myricetin,
Q: Quercetin,
K: Kaempferol,
Tri: Tricetin,
Lut: Luteolin,
Api: Apigenin
Del (%): Proportion of delphinidin in total anthocyanidins

### Example 5

### Transfer of Pansy F3′5′H#40 Gene, Torenia Flavone Synthase Gene and Torenia Anthocyanin Methyltransferase Gene into Rose Variety “WKS124”

Plasmid pSPB1309 described in Example 3 was treated with PacI for cleavage at the PacI site present near the linkage point between the torenia flavone synthase expression cassette and the pansy F3′5′H#40 gene expression cassette (more specifically, located near the 3′-end of the D8 terminator of the flavone synthase expression cassette) and at the PacI site in the vector multicloning site, to cut out the pansy F3′5′H#40 gene expression cassette.

Separately, the binary vector pSPB1530 having the torenia methyltransferase gene expression cassette, described in WO2003-062428, was cut with PacI and the aforementioned pansy F3′5′H#40 expression cassette was inserted therein in the same direction as the methyltransferase gene expression cassette. This plasmid was designated as TMT-BP40.

Separately, plasmid pSPB1309 was cleaved with AscI to cut out the torenia flavone synthase expression cassette. This was inserted into the AscI site of TMT-BP40 in the same direction as the previous expression cassettes, and the obtained plasmid was designated as pSFL535. This plasmid constitutively expresses the pansy F3′5′H#40 gene, the torenia methyltransferase gene and the torenia flavone synthase gene in plants.

Plasmid pSFL535 obtained in this manner was transferred into the salmon-pink rose variety “WKS124”, and 173 transformants were obtained. Accumulation of malvidin (an anthocyanidin that has been methylated at the 3′ and 5′ positions of delphinidin) was confirmed in 88 of 98 anthocyanidin-analyzed transformants, and the presence of product indicated that the pansy F3′5′H#40 gene and torenia anthocyanin methyltransferase gene were functioning in the rose petals. The malvidin content was a maximum of 84% (average: 50%).

Also, novel accumulation of flavones (tricetin, luteolin and apigenin) was confirmed in 77 transformants, due to the action of the torenia flavone synthase gene. At maximum, the total amount of flavones was a high content of 4.58 mg per 1 g of fresh petal weight. Methylated tricetin was detected in 51 transformants.

The analysis values for representative transformants are shown in Table 6 below.

TABLE 6

| | | | Anthocyanidins | | | | | | Flavonols | | | Flavones | | | |
|---|---|---|---|---|---|---|---|---|---|---|---|---|---|---|---|
| Plant | Del* | Mal | (mg/g) | | | | | | (mg/g) | | | (mg/g) | | | |
| No. | (%) | (%) | Del | Cya | Pet | Pel | Peo | Mal | M | Q | K | Tri | Lut | Api | Total |
| Control | 0.0 | 0.0 | 0.000 | 0.006 | 0.000 | 0.073 | 0.000 | 0.000 | 0.000 | 0.076 | 3.312 | 0.000 | 0.000 | 0.000 | 0.00 |
| 1 | 97.8 | 65.0 | 0.121 | 0.005 | 0.079 | 0.000 | 0.009 | 0.397 | 0.331 | 0.000 | 0.000 | 2.273 | 0.623 | 0.207 | 3.103 |
| 2 | 96.9 | 81.3 | 0.048 | 0.005 | 0.048 | 0.000 | 0.014 | 0.500 | 0.231 | 0.000 | 0.000 | 3.699 | 0.762 | 0.116 | 4.577 |
| 3 | 96.4 | 83.8 | 0.014 | 0.003 | 0.024 | 0.000 | 0.008 | 0.258 | 0.209 | 0.009 | 0.510 | 1.334 | 0.343 | 0.538 | 2.215 |
| 4 | 87.4 | 77.9 | 0.008 | 0.026 | 0.017 | 0.000 | 0.007 | 0.208 | 0.020 | 0.000 | 0.000 | 3.651 | 0.451 | 0.087 | 4.188 |
| 5 | 93.2 | 79.9 | 0.011 | 0.010 | 0.019 | 0.000 | 0.005 | 0.182 | 0.062 | 0.000 | 0.000 | 3.011 | 0.278 | 0.000 | 3.289 |
| 6 | 93.2 | 61.2 | 0.160 | 0.014 | 0.113 | 0.002 | 0.042 | 0.521 | 0.279 | 0.000 | 0.405 | 1.329 | 0.448 | 0.616 | 2.393 |
| 7 | 90.9 | 63.5 | 0.071 | 0.010 | 0.048 | 0.002 | 0.028 | 0.275 | 0.102 | 0.000 | 0.145 | 0.765 | 0.299 | 0.403 | 1.468 |
| 8 | 95.1 | 64.7 | 0.165 | 0.012 | 0.121 | 0.002 | 0.033 | 0.610 | 0.280 | 0.000 | 0.116 | 1.700 | 0.503 | 0.465 | 2.667 |
| 9 | 86.7 | 67.5 | 0.031 | 0.006 | 0.033 | 0.008 | 0.030 | 0.225 | 0.071 | 0.000 | 0.579 | 1.217 | 0.186 | 0.980 | 2.383 |
| 10 | 93.1 | 72.7 | 0.070 | 0.008 | 0.067 | 0.002 | 0.036 | 0.486 | 0.126 | 0.000 | 0.176 | 1.858 | 0.459 | 0.545 | 2.861 |
| 11 | 85.2 | 60.9 | 0.112 | 0.064 | 0.065 | 0.003 | 0.041 | 0.443 | 0.188 | 0.000 | 0.221 | 1.478 | 0.397 | 0.530 | 2.405 |
| 12 | 93.2 | 67.1 | 0.099 | 0.009 | 0.075 | 0.001 | 0.036 | 0.447 | 0.053 | 0.000 | 0.023 | 1.472 | 0.297 | 0.058 | 1.826 |
| 13 | 89.2 | 64.3 | 0.072 | 0.015 | 0.070 | 0.002 | 0.045 | 0.367 | 0.108 | 0.000 | 0.064 | 1.473 | 0.310 | 0.108 | 1.891 |
| 14 | 90.2 | 63.3 | 0.082 | 0.016 | 0.080 | 0.003 | 0.040 | 0.383 | 0.070 | 0.344 | 0.094 | 1.348 | 0.308 | 0.148 | 1.803 |
| 15 | 87.8 | 64.4 | 0.035 | 0.011 | 0.036 | 0.001 | 0.025 | 0.196 | 0.150 | 0.000 | 0.099 | 1.863 | 0.358 | 0.075 | 2.296 |
| 16 | 92.0 | 70.7 | 0.061 | 0.009 | 0.055 | 0.002 | 0.033 | 0.383 | 0.113 | 0.000 | 0.067 | 2.389 | 0.421 | 0.237 | 3.046 |
| 17 | 91.1 | 65.2 | 0.140 | 0.019 | 0.117 | 0.003 | 0.066 | 0.648 | 0.313 | 0.000 | 0.191 | 2.727 | 0.565 | 0.133 | 3.425 |
| 18 | 90.0 | 63.8 | 0.056 | 0.010 | 0.044 | 0.001 | 0.028 | 0.245 | 0.161 | 0.000 | 0.139 | 0.963 | 0.212 | 0.056 | 1.231 |
| 19 | 89.3 | 68.3 | 0.065 | 0.013 | 0.067 | 0.004 | 0.051 | 0.430 | 0.076 | 0.000 | 0.042 | 2.438 | 0.353 | 0.134 | 2.924 |
| 20 | 89.1 | 63.8 | 0.060 | 0.015 | 0.049 | 0.002 | 0.030 | 0.277 | 0.224 | 0.041 | 0.208 | 1.484 | 0.324 | 0.215 | 2.022 |

Control: WKS124 control
Del: Delphinidin,
Cya: Cyanidin,
Pet: Petunidin,
Pel: Pelargonidin,
Peo: Peonidin,
Mal: Malvidin,
M: Myricetin,
Q: Quercetin,
K: Kaempferol,
Tri: Tricetin,
Lut: Luteolin,
Api: Apigenin
Del (%): Proportion of delphinidinic pigments (delphinidin, petunidin, malvidin) in total anthocyanins,
Mal (%): Proportion of malvidin in total anthocyanidins

### Example 6

### Transfer of Pansy F3′5′H#40 Gene, Torenia Flavone Synthase Gene and Torenia Anthocyanin Methyltransferase Gene into Rose Variety “Lavande”

Plasmid pSFL535 described in Example 5 was transferred into the mauve rose variety “Lavande”, and 130 transformants were obtained. Accumulation of malvidin (an anthocyanidin that has been methylated at the 3′ and 5′ positions of delphinidin) was confirmed in 37 of 118 anthocyanidin-analyzed transformants, and the presence of product indicated that the pansy F3′5′H#40 gene and torenia anthocyanin methyltransferase gene were functioning in the rose petals. The malvidin content was a maximum of 55.6% (average: 20.5%).

Also, novel accumulation of flavones (tricetin, luteolin and apigenin) was confirmed in 78 transformants, due to the action of the torenia flavone synthase gene. At maximum, the total amount of flavones was a high content of 5.11 mg per 1 g of fresh petal weight. In addition, methylated tricetin or luteolin was detected in 20 of the flavone-producing transformants.

The analysis values for representative transformants are shown in Table 7 below.

TABLE 7

| Plant | Del | Mal | Anthocyanidins (mg/g) | | | | | | Flavonols (mg/g) | | | Flavones(mg/g) | | | |
|---|---|---|---|---|---|---|---|---|---|---|---|---|---|---|---|
| No. | (%) | (%) | Del | Cya | Pet | Pel | Peo | Mal | M | Q | K | Tri | Lut | Api | Total |
| Control | 0% | 0% | 0.000 | 0.109 | 0.000 | 0.000 | 0.000 | 0.000 | 0.000 | 1.020 | 0.195 | 0.000 | 0.000 | 0.000 | 0.000 |
| (Lavande) | | | | | | | | | | | | | | | |
| 1 | 20.7% | 3.3% | 0.012 | 0.065 | 0.002 | 0.001 | 0.002 | 0.003 | 0.097 | 0.272 | 0.015 | 0.289 | 0.035 | 0.000 | 0.324 |
| 2 | 45.9% | 31.8% | 0.003 | 0.006 | 0.001 | 0.000 | 0.007 | 0.007 | 0.024 | 0.076 | 0.000 | 1.062 | 0.212 | 0.009 | 1.283 |
| 3 | 74.8% | 46.5% | 0.063 | 0.023 | 0.010 | 0.000 | 0.041 | 0.119 | 0.458 | 0.285 | 0.049 | 0.204 | 0.022 | 0.000 | 0.226 |
| 4 | 71.4% | 51.6% | 0.018 | 0.010 | 0.005 | 0.000 | 0.022 | 0.058 | 0.292 | 0.153 | 0.011 | 0.139 | 0.015 | 0.000 | 0.153 |
| 5 | 70.5% | 32.1% | 0.025 | 0.012 | 0.007 | 0.000 | 0.012 | 0.027 | 0.510 | 0.192 | 0.033 | 0.000 | 0.026 | 0.000 | 0.026 |
| 6 | 28.9% | 4.4% | 0.031 | 0.096 | 0.005 | 0.000 | 0.005 | 0.006 | 0.268 | 0.619 | 0.037 | 0.262 | 0.038 | 0.009 | 0.310 |
| 7 | 84.4% | 53.4% | 0.036 | 0.008 | 0.014 | 0.000 | 0.017 | 0.086 | 0.811 | 0.168 | 0.000 | 1.054 | 0.086 | 0.000 | 1.139 |
| 8 | 79.8% | 53.2% | 0.032 | 0.011 | 0.012 | 0.000 | 0.022 | 0.087 | 0.316 | 0.107 | 0.004 | 0.863 | 0.037 | 0.000 | 0.900 |
| 9 | 83.3% | 55.6% | 0.038 | 0.012 | 0.012 | 0.006 | 0.012 | 0.100 | 0.593 | 0.013 | 0.000 | 4.885 | 0.223 | 0.000 | 5.108 |
| 10 | 63.0% | 33.6% | 0.003 | 0.002 | 0.000 | 0.001 | 0.001 | 0.003 | 0.032 | 0.000 | 0.000 | 3.992 | 0.219 | 0.003 | 4.214 |
| 11 | 88.1% | 45.8% | 0.027 | 0.004 | 0.006 | 0.000 | 0.005 | 0.036 | 0.285 | 0.060 | 0.009 | 2.779 | 0.077 | 0.000 | 2.855 |
| 12 | 86.2% | 43.4% | 0.041 | 0.009 | 0.011 | 0.000 | 0.008 | 0.053 | 0.412 | 0.103 | 0.020 | 4.243 | 0.119 | 0.000 | 4.363 |
| 13 | 73.6% | 38.0% | 0.019 | 0.009 | 0.004 | 0.000 | 0.009 | 0.025 | 0.227 | 0.049 | 0.000 | 3.794 | 0.000 | 0.000 | 3.794 |
| 14 | 89.3% | 49.7% | 0.035 | 0.005 | 0.007 | 0.000 | 0.006 | 0.052 | 0.217 | 0.030 | 0.000 | 4.936 | 0.139 | 0.000 | 5.075 |
| 15 | 65.1% | 31.1% | 0.010 | 0.007 | 0.005 | 0.000 | 0.007 | 0.013 | 0.155 | 0.050 | 0.000 | 3.184 | 0.128 | 0.000 | 3.312 |

Control: Lavande control
Del: Delphinidin,
Cya: Cyanidin,
Pet: Petunidin,
Pel: Pelargonidin:
Peo: Peonidin,
Mal: Malvidin,
M: Myricetin,
Q: Quercetin:
K: Kaempferol,
Tri: Tricetin,
Lut: Luteolin,
Api: Apigenin
Del (%): Proportion of delphinidinic pigments (delphinidin, petunidin, malvidin) in total anthocyanins
Mal (%): Proportion of malvidin in total anthocyanidins

### Example 7

### Transfer of Pansy F3′5′H#40 Gene, Torenia Flavone Synthase Gene and Torenia Anthocyanin Methyltransferase Gene into Rose Variety “WKS82”

Plasmid pSFL535 described in Example 5 was transferred into the mauve rose variety “WKS82”, and 250 transformants were obtained. Accumulation of malvidin (an anthocyanidin that has been methylated at the 3′ and 5′ positions of delphinidin) was confirmed in 110 of 232 anthocyanidin-analyzed transformants, and the presence of product indicated that the pansy F3′5′H#40 gene and torenia anthocyanin methyltransferase gene were functioning in the rose petals. The malvidin content was a maximum of 65.2% (average: 19.7%).

Also, novel accumulation of flavones (tricetin, luteolin and apigenin) was confirmed in 125 transformants, due to the action of the torenia flavone synthase gene. At maximum, the total amount of flavones was a high content of 4.71 mg per 1 g of fresh petal weight. In addition, methylated tricetin or luteolin was detected in 80 of the flavone-producing transformants.

The analysis values for representative transformants are shown in Table 8 below.

TABLE 8

| Plant | Del | Mal | Anthocyanidins (mg/g) | | | | | | Flavonols (mg/g) | | | Flavones(mg/g) | | | |
|---|---|---|---|---|---|---|---|---|---|---|---|---|---|---|---|
| No. | (%) | (%) | Del | Cya | Pet | Pel | Peo | Mal | M | Q | K | Tri | Lut | Api | Total |
| Control | 0% | 0% | 0.000 | 0.124 | 0.000 | 0.000 | 0.000 | 0.000 | 0.000 | 1.598 | 0.081 | 0.000 | 0.000 | 0.000 | 0.000 |
| (WKS82) | | | | | | | | | | | | | | | |
| 1 | 57.5% | 46.1% | 0.003 | 0.006 | 0.003 | 0.000 | 0.018 | 0.026 | 0.494 | 0.750 | 0.064 | 0.764 | 0.000 | 0.000 | 0.764 |
| 2 | 70.5% | 51.0% | 0.007 | 0.005 | 0.004 | 0.000 | 0.011 | 0.028 | 0.564 | 0.384 | 0.055 | 2.977 | 0.199 | 0.000 | 3.176 |
| 3 | 82.1% | 65.2% | 0.006 | 0.004 | 0.005 | 0.000 | 0.008 | 0.042 | 0.800 | 0.536 | 0.115 | 0.534 | 0.000 | 0.000 | 0.534 |
| 4 | 75.3% | 57.5% | 0.004 | 0.003 | 0.003 | 0.000 | 0.008 | 0.024 | 0.387 | 0.288 | 0.074 | 1.808 | 0.160 | 0.000 | 1.968 |
| 5 | 55.2% | 37.6% | 0.005 | 0.009 | 0.004 | 0.000 | 0.015 | 0.020 | 1.054 | 0.806 | 0.038 | 0.114 | 0.000 | 0.000 | 0.114 |
| 6 | 48.8% | 37.8% | 0.004 | 0.006 | 0.002 | 0.000 | 0.021 | 0.020 | 0.700 | 1.319 | 0.148 | 0.034 | 0.000 | 0.000 | 0.034 |
| 7 | 78.4% | 62.8% | 0.007 | 0.003 | 0.004 | 0.000 | 0.011 | 0.042 | 0.577 | 0.266 | 0.015 | 0.302 | 0.022 | 0.000 | 0.324 |
| 8 | 54.6% | 39.1% | 0.006 | 0.009 | 0.003 | 0.000 | 0.018 | 0.023 | 0.571 | 0.774 | 0.045 | 0.172 | 0.028 | 0.000 | 0.200 |
| 9 | 73.5% | 57.3% | 0.009 | 0.004 | 0.004 | 0.000 | 0.016 | 0.044 | 0.866 | 0.511 | 0.031 | 0.104 | 0.000 | 0.000 | 0.104 |
| 10 | 75.9% | 57.5% | 0.005 | 0.002 | 0.002 | 0.000 | 0.007 | 0.022 | 0.882 | 0.498 | 0.151 | 0.038 | 0.000 | 0.000 | 0.038 |
| 11 | 69.3% | 52.9% | 0.007 | 0.006 | 0.005 | 0.000 | 0.016 | 0.038 | 0.825 | 0.411 | 0.029 | 0.095 | 0.000 | 0.000 | 0.095 |
| 12 | 71.4% | 50.2% | 0.013 | 0.007 | 0.006 | 0.000 | 0.020 | 0.046 | 0.721 | 0.459 | 0.022 | 0.075 | 0.004 | 0.000 | 0.080 |
| 13 | 59.8% | 42.2% | 0.016 | 0.014 | 0.009 | 0.000 | 0.044 | 0.062 | 1.540 | 1.415 | 0.202 | 0.193 | 0.000 | 0.000 | 0.095 |
| 14 | 67.9% | 50.9% | 0.006 | 0.006 | 0.006 | 0.000 | 0.017 | 0.036 | 0.829 | 0.704 | 0.125 | 0.000 | 0.000 | 0.000 | 0.200 |
| 15 | 34.4% | 13.0% | 0.006 | 0.014 | 0.003 | 0.000 | 0.013 | 0.006 | 0.230 | 1.109 | 0.000 | 4.155 | 0.551 | 0.006 | 4.711 |

Control: WKS82 control
Del: Delphinidin,
Cya: Cyanidin,
Pet: Petunidin,
Pel: Pelargonidin:
Peo: Peonidin,
Mal: Malvidin,
M: Myricetin,
Q: Quercetin:
K: Kaempferol,
Tri: Tricetin,
Lut: Luteolin,
Api: Apigenin
Del (%): Proportion of delphinidinic pigments (delphinidin, petunidin, malvidin) in total anthocyanins
Mal (%): Proportion of malvidin in total anthocyanidins

### Example 8

### Transfer of Pansy F3′5′H#40 Gene, Torenia Flavone Synthase Gene and Torenia Anthocyanin Methyltransferase Gene into Rose Variety “WKS140”

Plasmid pSFL535 described in Example 5 was transferred into the mauve rose variety “WKS140”, and 74 transformants were obtained. Accumulation of malvidin (an anthocyanidin that has been methylated at the 3′ and 5′ positions of delphinidin) was confirmed in 20 of 74 anthocyanidin-analyzed transformants, and the presence of product indicated that the pansy F3′5′H#40 gene and torenia anthocyanin methyltransferase gene were functioning in the rose petals. The malvidin content was a maximum of 51.3% (average: 33.5%).

Also, novel accumulation of flavones (tricetin, luteolin and apigenin) was confirmed in 29 transformants, due to the action of the torenia flavone synthase gene. At maximum, the total amount of flavones was a high content of 3.04 mg per 1 g of fresh petal weight. In addition, methylated tricetin or luteolin was detected in 20 of the flavone-producing transformants.

The analysis values for representative transformants are shown in Table 9 below.

TABLE 9

| Plant | Del | Mal | Anthocyanidins (mg/g) | | | | | | Flavonols (mg/g) | | | Flavones(mg/g) | | | |
|---|---|---|---|---|---|---|---|---|---|---|---|---|---|---|---|
| No. | (%) | (%) | Del | Cya | Pet | Pel | Peo | Mal | M | Q | K | Tri | Lut | Api | Total |
| Control | 0% | 0% | 0.000 | 0.075 | 0.000 | 0.000 | 0.000 | 0.000 | 0.000 | 2.412 | 0.271 | 0.000 | 0.000 | 0.000 | 0.000 |
| (WKS140) | | | | | | | | | | | | | | | |
| 1 | 62.0% | 31.7% | 0.025 | 0.020 | 0.015 | 0.000 | 0.030 | 0.042 | 0.655 | 1.085 | 0.202 | 2.314 | 0.305 | 0.032 | 2.650 |
| 2 | 67.3% | 38.3% | 0.013 | 0.009 | 0.009 | 0.000 | 0.015 | 0.029 | 0.491 | 0.627 | 0.104 | 1.790 | 0.227 | 0.031 | 2.048 |
| 3 | 79.6% | 34.1% | 0.025 | 0.008 | 0.011 | 0.000 | 0.008 | 0.027 | 0.572 | 0.555 | 0.129 | 2.388 | 0.237 | 0.015 | 2.639 |
| 4 | 69.8% | 38.9% | 0.021 | 0.012 | 0.011 | 0.000 | 0.019 | 0.040 | 0.589 | 0.766 | 0.165 | 1.941 | 0.282 | 0.014 | 2.237 |
| 5 | 80.4% | 51.3% | 0.013 | 0.005 | 0.009 | 0.000 | 0.010 | 0.038 | 0.513 | 0.307 | 0.074 | 1.392 | 0.166 | 0.018 | 1.577 |
| 6 | 70.1% | 35.8% | 0.014 | 0.008 | 0.006 | 0.000 | 0.010 | 0.021 | 0.607 | 0.538 | 0.108 | 1.297 | 0.177 | 0.019 | 1.493 |
| 7 | 67.2% | 34.7% | 0.020 | 0.013 | 0.009 | 0.000 | 0.017 | 0.031 | 1.005 | 0.717 | 0.127 | 1.805 | 0.264 | 0.028 | 2.097 |
| 8 | 70.0% | 36.2% | 0.019 | 0.010 | 0.009 | 0.000 | 0.015 | 0.029 | 0.831 | 0.802 | 0.143 | 1.909 | 0.241 | 0.027 | 2.176 |
| 9 | 70.9% | 37.9% | 0.015 | 0.008 | 0.008 | 0.000 | 0.012 | 0.027 | 0.497 | 0.690 | 0.106 | 1.841 | 0.265 | 0.032 | 2.138 |
| 10 | 69.9% | 36.0% | 0.018 | 0.010 | 0.009 | 0.000 | 0.015 | 0.030 | 0.544 | 0.663 | 0.143 | 2.102 | 0.236 | 0.017 | 2.355 |
| 11 | 57.4% | 31.0% | 0.011 | 0.011 | 0.008 | 0.000 | 0.019 | 0.022 | 0.386 | 0.892 | 0.129 | 2.088 | 0.271 | 0.012 | 2.372 |
| 12 | 62.9% | 32.4% | 0.016 | 0.014 | 0.010 | 0.000 | 0.018 | 0.028 | 0.351 | 0.846 | 0.114 | 2.274 | 0.281 | 0.009 | 2.565 |
| 13 | 62.1% | 34.1% | 0.021 | 0.018 | 0.014 | 0.000 | 0.030 | 0.042 | 0.887 | 0.789 | 0.177 | 1.855 | 0.389 | 0.018 | 2.262 |
| 14 | 73.7% | 37.5% | 0.016 | 0.006 | 0.004 | 0.000 | 0.008 | 0.021 | 0.597 | 0.489 | 0.081 | 1.664 | 0.158 | 0.000 | 1.821 |
| 15 | 58.6% | 28.3% | 0.013 | 0.012 | 0.007 | 0.000 | 0.016 | 0.019 | 0.513 | 1.121 | 0.166 | 2.650 | 0.373 | 0.015 | 3.038 |

Control: WKS140 control
Del: Delphinidin,
Cya: Cyanidin,
Pet: Petunidin,
Pel: Pelargonidin:
Peo: Peonidin,
Mal: Malvidin,
M: Myricetin,
Q: Quercetin:
K: Kaempferol,
Tri: Tricetin,
Lut: Luteolin,
Api: Apigenin
Del (%): Proportion of delphinidinic pigments (delphinidin, petunidin, malvidin) in total anthocyanins
Mal (%): Proportion of malvidin in total anthocyanidins

### Example 9

### Propagation of Flavone and Malvidin Synthesis Ability to Progeny—Hybridization Between Cultivated Roses and Gene Recombinant Roses Containing Transferred Pansy F3′5′H#40 Gene, Torenia Flavone Synthase Gene and Torenia Anthocyanin Methyltransferase Gene

In order to investigate the mode of inheritance to progeny for flavone synthesis ability in roses, cross-breeding was carried out using a malvidin- and flavone-producing rose created in Example 5 (plant No. 6 in Table 6) as the pollen parent. As the seed parent there was used the medium-sized cultivated rose “Medeo” (floribunda rose variety “Medeo”).

Accumulation of malvidin was confirmed in 7 of the pigment-analyzed transformant F1 hybrid progeny that were obtained, and the presence of product indicated that the pansy F3′5′H#40 gene and torenia anthocyanin methyltransferase gene were functioning in the rose petals. The malvidin content was a maximum of 68.2% (average: 46.6%).

On the other hand, novel accumulation of flavones (tricetin, luteolin and apigenin) was confirmed in 8 transformant progeny, due to the action of the torenia flavone synthase gene. At maximum, the total amount of flavones was an extremely high content of 7.35 mg per 1 g of fresh petal weight. In addition, methylated tricetin or luteolin was detected in 6 of the flavone-producing transformant progeny.

The analysis values for representative transformant progeny are shown in Table 10 below.

TABLE 10

| Plant | Del | Mal | Anthocyanidins (mg/g) | | | | | | Flavonols (mg/g) | | | Flavones(mg/g) | | | |
|---|---|---|---|---|---|---|---|---|---|---|---|---|---|---|---|
| No. | (%) | (%) | Del | Cya | Pet | Pel | Peo | Mal | M | Q | K | Tri | Lut | Api | Total |
| Pollen | 93.2% | 61.2% | 0.160 | 0.014 | 0.113 | 0.002 | 0.042 | 0.521 | 0.279 | 0.000 | 0.405 | 1.329 | 0.448 | 0.616 | 2.393 |
| parent | | | | | | | | | | | | | | | |
| (Example 5 | | | | | | | | | | | | | | | |
| Plant No. 6) | | | | | | | | | | | | | | | |
| Seed parent | 0% | 0% | 0.000 | 0.004 | 0.000 | 0.004 | 0.000 | 0.000 | 0.000 | 0.028 | 2.323 | 0.000 | 0.000 | 0.000 | 0.000 |
| (var. Medeo) | | | | | | | | | | | | | | | |
| 1 | 0.0% | 0.0% | 0.000 | 0.015 | 0.000 | 0.122 | 0.000 | 0.000 | 0.000 | 0.000 | 4.318 | 0.000 | 0.000 | 0.000 | 0.000 |
| 2 | 82.6% | 49.1% | 0.165 | 0.034 | 0.085 | 0.005 | 0.090 | 0.367 | 0.311 | 0.039 | 0.118 | 1.596 | 0.064 | 0.006 | 1.666 |
| 3 | 0.0% | 0.0% | 0.000 | 0.001 | 0.000 | 0.004 | 0.000 | 0.000 | 0.000 | 0.000 | 2.391 | 0.000 | 0.000 | 0.000 | 0.000 |
| 4 | 80.1% | 50.5% | 0.073 | 0.028 | 0.064 | 0.00 | 0.054 | 0.233 | 0.210 | 0.048 | 0.429 | 0.000 | 0.000 | 0.032 | 0.032 |
| 5 | 94.4% | 52.8% | 0.003 | 0.001 | 0.003 | 0.000 | 0.000 | 0.009 | 0.408 | 0.069 | 0.668 | 2.024 | 0.152 | 0.222 | 2.398 |
| 6 | 81.8% | 34.4% | 0.056 | 0.015 | 0.034 | 0.002 | 0.017 | 0.065 | 0.076 | 0.033 | 0.202 | 3.860 | 0.123 | 0.039 | 4.023 |
| 7 | 48.2% | 0.0% | 0.011 | 0.002 | 0.011 | 0.001 | 0.021 | 0.000 | 0.089 | 0.038 | 0.808 | 1.603 | 0.117 | 0.217 | 1.937 |
| 8 | 90.6% | 35.5% | 0.107 | 0.016 | 0.071 | 0.002 | 0.013 | 0.114 | 0.080 | 0.010 | 0.100 | 6.497 | 0.351 | 0.504 | 7.351 |
| 9 | 70.4% | 35.8% | 0.008 | 0.003 | 0.001 | 0.002 | 0.003 | 0.009 | 0.118 | 0.038 | 0.523 | 2.902 | 0.137 | 0.048 | 3.088 |
| 10 | 91.2% | 68.2% | 0.011 | 0.002 | 0.012 | 0.001 | 0.007 | 0.068 | 1.131 | 0.324 | 1.077 | 1.031 | 0.091 | 0.033 | 1.154 |

Del: Delphinidin,
Cya: Cyanidin,
Pet: Petunidin,
Pel: Pelargonidin:
Peo: Peonidin,
Mal: Malvidin,
M: Myricetin,
Q: Quercetin:
K: Kaempferol,
Tri: Tricetin,
Lut: Luteolin,
Api: Apigenin
Del (%): Proportion of delphinidinic pigments (delphinidin, petunidin, malvidin) in total anthocyanins
Mal (%): Proportion of malvidin in total anthocyanidins

### Example 10

### Propagation of Flavone Synthesis Ability to Progeny

Cross-Breeding of Rose Variety “WKS124” Containing Transferred Pansy F3′5′H#40 Gene and Torenia Anthocyanin Methyltransferase Gene, with Rose Variety “Lavande” Containing Transferred Pansy F3′5′H#40 Gene and Torenia Flavone Synthase Gene.

In order to investigate the mode of inheritance to progeny for flavone synthesis ability in roses, cross-breeding was carried out using a flavone-producing line created in Example 3 (plant No. 4 in Table 4) as the pollen parent. As the seed parent there was used transformant WKS124/1532-12-1 (described in WO2003/062428), with high accumulation of malvidin in the petals due to transfer of pSPB1532 into the rose variety WKS124 and the resulting actions of the pansy F3′5′H#40 gene and torenia anthocyanin methyltransferase gene.

Upon pigment analysis of 149 of the obtained transformant progeny, accumulation of flavones (tricetin, luteolin, apigenin) was confirmed in 88 individuals. At maximum, the total amount of flavones was a high content of 4.09 mg per 1 g of fresh petal weight. Also, methylated tricetin was detected in 42 progeny, while methylated luteolin (chrysoeriol (3′-Met-Lut)) was detected in 11. Accumulation of malvidin was confirmed in 129 of the 149 pigment-analyzed progeny. The malvidin content was a maximum of 79% (average: 36%).

The analysis values for representative transformant progeny are shown in Table 11 below.

TABLE 11

| | | | Anthocyanidins | | | | | | Flavonols | | | Flavones | | | |
|---|---|---|---|---|---|---|---|---|---|---|---|---|---|---|---|
| Plant | Del* | Mal | (mg/g) | | | | | | (mg/g) | | | (mg/g) | | | |
| No. | (%) | (%) | Del | Cya | Pet | Pel | Peo | Mal | M | Q | K | Tri | Lut | Api | Total |
| Pollen | 44.9 | 0.0 | 0.031 | 0.038 | 0.000 | 0.000 | 0.000 | 0.000 | 0.000 | 0.359 | 0.027 | 0.000 | 1.684 | 0.000 | 1.684 |
| parent | | | | | | | | | | | | | | | |
| Seed | 93.2 | 73.0 | 0.127 | 0.011 | 0.112 | 0.003 | 0.066 | 0.863 | 0.365 | 0.093 | 0.348 | 0.000 | 0.000 | 0.000 | 0.000 |
| parent | | | | | | | | | | | | | | | |
| 1 | 92.1 | 69.1 | 0.032 | 0.005 | 0.030 | 0.000 | 0.016 | 0.186 | 0.197 | 0.105 | 0.090 | 1.950 | 0.078 | 0.059 | 2.088 |
| 2 | 75.3 | 56.7 | 0.076 | 0.048 | 0.055 | 0.005 | 0.121 | 0.400 | 0.345 | 0.081 | 0.097 | 2.879 | 0.156 | 0.086 | 3.121 |
| 3 | 80.6 | 60.6 | 0.041 | 0.015 | 0.039 | 0.004 | 0.059 | 0.244 | 0.000 | 0.000 | 0.113 | 2.986 | 0.193 | 0.000 | 3.179 |
| 4 | 82.4 | 65.8 | 0.005 | 0.002 | 0.006 | 0.000 | 0.009 | 0.043 | 0.000 | 0.131 | 0.084 | 2.036 | 0.066 | 0.000 | 2.103 |
| 5 | 68.8 | 56.7 | 0.010 | 0.006 | 0.012 | 0.013 | 0.036 | 0.101 | 0.000 | 0.093 | 0.179 | 1.740 | 0.224 | 0.000 | 1.965 |
| 6 | 79.3 | 60.4 | 0.018 | 0.009 | 0.017 | 0.000 | 0.029 | 0.111 | 0.089 | 0.053 | 0.084 | 1.956 | 0.093 | 0.029 | 2.078 |
| 7 | 86.1% | 52.1 | 0.158 | 0.044 | 0.125 | 0.003 | 0.069 | 0.432 | 0.000 | 0.118 | 0.300 | 3.059 | 0.363 | 0.397 | 3.819 |
| 8 | 81.3 | 59.8 | 0.026 | 0.010 | 0.027 | 0.011 | 0.025 | 0.149 | 0.000 | 0.247 | 0.226 | 1.489 | 0.232 | 0.179 | 1.900 |
| 9 | 79.2 | 59.2% | 0.015 | 0.008 | 0.014 | 0.000 | 0.022 | 0.086 | 0.422 | 0.398 | 0.224 | 2.510 | 0.726 | 0.094 | 3.330 |
| 10 | 82.5 | 66.8% | 0.019 | 0.008 | 0.022 | 0.000 | 0.038 | 0.175 | 0.445 | 0.559 | 0.322 | 2.122 | 0.446 | 0.111 | 2.678 |
| 11 | 73.3 | 59.3 | 0.036 | 0.025 | 0.037 | 0.000 | 0.112 | 0.306 | 0.121 | 0.130 | 0.000 | 0.596 | 0.565 | 0.066 | 1.227 |
| 12 | 94.0 | 76.2 | 0.018 | 0.002 | 0.018 | 0.000 | 0.010 | 0.154 | 0.840 | 0.426 | 0.445 | 3.655 | 0.306 | 0.124 | 4.086 |
| 13 | 82.8 | 62.8 | 0.009 | 0.004 | 0.010 | 0.000 | 0.012 | 0.059 | 0.394 | 0.400 | 0.278 | 1.068 | 0.250 | 0.096 | 1.414 |
| 14 | 82.7 | 58.8 | 0.119 | 0.048 | 0.122 | 0.011 | 0.115 | 0.592 | 0.327 | 0.089 | 0.074 | 1.940 | 0.131 | 0.085 | 2.156 |
| 15 | 78.4 | 61.6 | 0.009 | 0.006 | 0.009 | 0.000 | 0.018 | 0.066 | 0.313 | 0.582 | 0.370 | 1.634 | 0.423 | 0.143 | 2.200 |

Del: Delphinidin,
Cya: Cyanidin,
Pet: Petunidin,
Pel: Pelargonidin,
Peo: Peonidin,
Mal: Malvidin,
M: Myricetin,
Q: Quercetin,
K: Kaempferol,
Tri: Tricetin,
Lut: Luteolin,
Api: Apigenin
Del (%): Proportion of delphinidinic pigments (delphinidin, petunidin, malvidin) in total anthocyanins
Mal (%): Proportion of malvidin in total anthocyanidins

### Example 11

### Evaluation of Flavone-Containing Rose Flower Color

The transformants created in Examples 4 and 5 (host: rose variety “WKS124”) were divided into 5 groups: (1) those accumulating delphinidin as the major pigment and containing no flavones, (2) those accumulating delphinidin as the major pigment and containing flavones, (3) those highly accumulating malvidin as the major pigment and containing no flavones, (4) those highly accumulating malvidin as the major pigment and containing flavones, and (5) host (accumulating pelargonidin as the major pigment), and the color shade of the petals were evaluated using a spectrocolorimeter (n=10).

In both the roses with delphinidin as the major pigment and the roses with malvidin as the major pigment, a shift in hue angle of the petals toward blue had occurred when flavones were copresent. This tendency was more pronounced in the roses with malvidin as the major pigment, and the reflection spectrum minimum (λMin) was also shifted significantly toward the long wavelength end. These results confirmed that the petal color shade had changed to blue by the copresence of flavones. The results are shown in Table 12 below.

TABLE 12

| | | Measured color values | |
|---|---|---|---|
| | | | Reflection spectrum |
| Gene/flavonoid composition | | Hue angle | minimum (λMin) |
| Host | No gene transfer (WKS124 control) | 31.14° | Average: 520 nm |
| | Pelargonidin accumulated as main | (=391.14°) | Maximum: 520 nm |
| | pigment, absolutely no flavones present | | |
| Ex. 7 | (1) Pansy F3′,5′H | Average: 349.03° | Average: 540 nm |
| | Delphinidin highly accumulated as main | Bluest value: 344.68° | Maximum: 540 nm |
| | pigment, absolutely no flavones present | | |
| | (2) Pansy F3′,5′H + torenia FNS | Average: 343.64° | Average: 540 nm |
| | Delphinidin highly accumulated as main | Bluest value: 337.18° | Maximum: 540 nm |
| | pigment, flavones present | | |
| | (3) Pansy F3′,5′H + torenia MT | Average: 341.78° | Average: 542 nm |
| | Malvidin highly accumulated as main | Bluest value: 336.82° | Maximum: 550 nm |
| | pigment, absolutely no flavones present | | |
| | (4) Pansy F3′,5′H + torenia MT + | Average: 334.45° | Average: 551 nm |
| | torenia FNS | Bluest value: 329.84° | Maximum: 560 nm |
| | Malvidin highly accumulated as main | | |
| | pigment, flavones present | | |

Hue angle (hue): The angle displacement for the color tone in the counter-clockwise direction from the a* (red direction) axis as 0° in the L*a*b* color system, for indication of the color position. An angle of 90° is the yellow direction, an angle of 180° is the green direction, an angle of 270° is the blue direction, and an angle of 0° (=360°) is the red direction. In other words, a numerical value approaching 270° represents a bluer color tone.

### INDUSTRIAL APPLICABILITY

According to the invention it is possible by genetic modification to add flavones and malvidin to roses, as popular flowering plants used for decoration, in order to alter rose flower color toward blue by a co-pigmentation effect. Roses with blue flower color are expected to be in high commercial demand as ornamental plants.

All of the patent documents and non-patent technical documents cited in the present specification are hereby incorporated by reference either individually or as a whole.

This completes the explanation of the invention, but the invention should be interpreted as encompassing any alterations or modifications such as do not deviate from the gist thereof, and the scope of the invention is not to be considered as based on the description in the examples but rather as defined by the scope of the attached claims.

1. A rose characterized by comprising a flavone and malvidin added by a genetic modification method.

2. A rose according to claim 1, which comprises a flavone and malvidin by expression of pansy (Viola x wittrockiana) flavonoid 3′,5′-hydroxylase and anthocyanin methyltransferase.

3. A rose according to claim 1, which comprises malvidin, a flavone and delphinidin by expression of an anthocyanin methyltransferase gene, a flavone synthase gene and the pansy (Viola x wittrockiana) flavonoid 3′,5′-hydroxylase gene.

4. A rose according to claim 1, wherein the flavone synthase gene is a flavone synthase gene derived from the family Scrophulariaceae.

5. A rose according to claim 4, wherein the flavone synthase gene derived from the family Scrophulariaceae is a flavone synthase gene derived from snapdragon of the family Scrophulariaceae (Scrophulariaceae, Antirrhinum majus).

6. A rose according to claim 4, wherein the flavone synthase gene derived from the family Scrophulariaceae is a flavone synthase gene derived from torenia of the family Scrophulariaceae (Scrophulariaceae, Torenia hybrida).

7. A rose according to claim 5, wherein the flavone synthase gene derived from snapdragon of the family Scrophulariaceae is a gene coding for
(1) flavone synthase having the amino acid sequence listed as SEQ ID NO: 2,
(2) flavone synthase having the amino acid sequence listed as SEQ ID NO: 2 modified by an addition or deletion of one or several amino acids and/or substitution of one or several amino acids by other amino acids,
(3) flavone synthase having an amino acid sequence with at least 90% sequence identity to the amino acid sequence listed as SEQ ID NO: 2, or
(4) flavone synthase encoded by nucleic acid that hybridizes with nucleic acid having the nucleotide sequence of SEQ ID NO: 1 under highly stringent conditions.

8. A rose according to claim 6, wherein the flavone synthase gene derived from torenia of the family Scrophulariaceae is a gene coding for
(1) flavone synthase having the amino acid sequence listed as SEQ ID NO: 4,
(2) flavone synthase having the amino acid sequence listed as SEQ ID NO: 4 modified by an addition or deletion of one or several amino acids and/or substitution of one or several amino acids by other amino acids,
(3) flavone synthase having an amino acid sequence with at least 90% sequence identity to the amino acid sequence listed as SEQ ID NO: 4, or
(4) flavone synthase encoded by nucleic acid that hybridizes with nucleic acid having the nucleotide sequence of SEQ ID NO: 3 under highly stringent conditions.

9. A rose according to claim 2, wherein the pansy flavonoid 3′,5′-hydroxylase gene is a gene coding for:
(1) flavonoid 3′,5′-hydroxylase having the amino acid sequence listed as SEQ ID NO: 8,
(2) flavonoid 3′,5′-hydroxylase having the amino acid sequence listed as SEQ ID NO: 8 modified by an addition or deletion of one or several amino acids and/or substitution of one or several amino acids by other amino acids,
(3) flavonoid 3′,5′-hydroxylase having an amino acid sequence with at least 90% sequence identity to the amino acid sequence listed as SEQ ID NO: 8, or
(4) flavonoid 3′,5′-hydroxylase encoded by nucleic acid that hybridizes with nucleic acid having the nucleotide sequence of SEQ ID NO: 7 under highly stringent conditions.

10. A rose according to claim 2, wherein the anthocyanin methyltransferase gene is a gene coding for:
(1) methyltransferase having the amino acid sequence listed as SEQ ID NO: 10,
(2) methyltransferase having the amino acid sequence listed as SEQ ID NO: 10 modified by an addition or deletion of one or several amino acids and/or substitution of one or several amino acids by other amino acids,
(3) methyltransferase having an amino acid sequence with at least 90% sequence identity to the amino acid sequence listed as SEQ ID NO: 10, or
(4) methyltransferase encoded by nucleic acid that hybridizes with nucleic acid having the nucleotide sequence of SEQ ID NO: 9 under highly stringent conditions.

11. A rose according to claim 2, wherein the flower color is changed with respect to the host before transfer of the anthocyanin methyltransferase gene, flavone synthase gene and pansy 3′,5′-hydroxylase gene.

12. A rose according to claim 11, wherein the change in flower color is a change toward blue.

13. A rose according to claim 11, wherein the change in flower color is a change such that the hue angle (θ) according to the L*a*b color system chromaticity diagram approaches 270° which is the blue axis.

14. A rose according to claim 11, wherein the change in flower color is a change such that the minimum value of the reflection spectrum of the petal shifts toward the longer wavelength end.

15. A rose portion, descendant, tissue, vegetative body or cell having the same properties as a rose according to claim 1.

16. A method for modifying the flower color of a rose by a co-pigmentation effect produced by adding a flavone and malvidin by a genetic modification technique.

17. The method according to claim 16, wherein the co-pigmentation effect is an effect of changing the flower color toward blue.

18. A rose according to claim 2, which comprises malvidin, a flavone and delphinidin by expression of an anthocyanin methyltransferase gene, a flavone synthase gene and the pansy (Viola x wittrockiana) flavonoid 3′,5′-hydroxylase gene.

19. A rose according to claim 12, wherein the change in flower color is a change such that the hue angle (θ) according to the L*a*b color system chromaticity diagram approaches 270° which is the blue axis.


SEQUENCE LISTING (10 sequences)

SEQ ID NO: 1 - DNA, 1706 bp, Antirrhinum majus
  CDS (40)..(1557): Nucleotide sequence encoding flavone synthase
gctttacacacacacacacacacacacacacaaacaaaaatgtctacacttgtctacagcacactcttcatcctctcaaccctcctcctcaccctcctaacccgcacccgccgcaagacccgcccgcccggcccattagccctccccttaataggccacttacacctcctcggcccaaagctccaccacaccttccaccaattctcccaacgctacggcccgctcatccagctctacctcggctccgtcccatgcgtcgtcgcttccacgcccgaactcgcccgcgaattcctcaagacgcacgaactcgacttctcgtcccgcaagcactccaccgccatcgacatcgtcacgtacgactcctcgttcgccttcgcgccgtacgggccgtactggaaattcatcaagaaattatgtacttacgagctactgggtgcccggaacttgagccatttccagcccattagagctttggaggtcaacagtttcttgagaattttgtacgagaaaacagagcagaaacagagtgttaatgtgactgaggagcttgtgaagctgacgagtaatgtgatcagtaacatgatgttggggatcaggtgttcggggacggaaggggaggcggaggtggcgaggacggtgataagggaggtgacgcagatatttggggagtttgatgtgtcggagattgtttggttttgtaagaatttggatctgcaggggattaggaagaggtcggaggatattaggaggaggtatgatgctttgttggagaagattattagtgatagggagaggttgaggttgagggggggtggtggtggagggggtggagaggtgaaggattttttggatatgttgttggatgtgatggagagtgagaaatcggaggtggagtttacgagggagcatctcaaagctttgattctggatttcttcactgccggtacagacacaacagcaatcacaacagaatgggcaatagcagaactcattagcaatccaaatgtactcaaaaaagctcaagaagagatggacaaagtcataggatcacaaaggttgttgcaagaatccgacgcccctaacttgccttacctcaacgcgatcataaaagaaacgttccgtctccaccctccaatccccatgctcactagaaaatcaatttctgacgttgtggtcaacgggtacacgatccctgccaaaacgctattgtttgtcaacctttggtccatgggaaggaatcctaactactgggaaaatccgatggagttccgacccgagaggtttctcgagaaagggaccgggtcgatagacgttaaagggcagcatttcgagttgctgccgtttggcacgggcaggcggggctgcccggggatgttgttaggcatgcaggagttgtttagtattatcggggctatggtgcagtgcttcgattggaaactgcccgatggtgtgaagtcggtcgacatgaccgagcggcccgggttgacggctccacgtgccaatgatttggtgtgccaattggtgccacggattgacccggtcgttgtctccggaccgtgaaccttaaggtagtatcgataatctgtttaattaaattgttatttgttgtgaggatttgatttttgttatgtatgattatgcgtggattaagataagcctgcaaggacaaattccctttctttgattgatgtcaatgagtttgtgtc

SEQ ID NO: 2 - protein, 506 aa, Antirrhinum majus
  Amino acid sequence of flavone synthase
MSTLVYSTLFILSTLLLTLLTRTRRKTRPPGPLALPLIGHLHLLGPKLHHTFHQFSQRYGPLIQLYLGSVPCVVASTPELAREFLKTHELDFSSRKHSTAIDIVTYDSSFAFAPYGPYWKFIKKLCTYELLGARNLSHFQPIRALEVNSFLRILYEKTEQKQSVNVTEELVKLTSNVISNMMLGIRCSGTEGEAEVARTVIREVTQIFGEFDVSEIVWFCKNLDLQGIRKRSEDIRRRYDALLEKIISDRERLRLRGGGGGGGGEVKDFLDMLLDVMESEKSEVEFTREHLKALILDFFTAGTDTTAITTEWAIAELISNPNVLKKAQEEMDKVIGSQRLLQESDAPNLPYLNAIIKETFRLHPPIPMLTRKSISDVVVNGYTIPAKTLLFVNLWSMGRNPNYWENPMEFRPERFLEKGTGSIDVKGQHFELLPFGTGRRGCPGMLLGMQELFSIIGAMVQCFDWKLPDGVKSVDMTERPGLTAPRANDLVCQLVPRIDPVVVSGP

SEQ ID NO: 3 - DNA, 1685 bp, Torenia hybrida
  CDS (57)..(1592): Nucleotide sequence encoding flavone synthase
atcgaaaccgctatatcattacatttacaacagcgctaaaaaaatatatataaagcatggacacagtcttaatcacactctacaccgccctgttcgtcatcaccaccaccttcctcctcctcctccgccgaaggggaccaccgtctccgcccggtcctctctccctacccataattggccacctccacctcctcggcccaagactccaccacacgttccatgaattctcactcaaatacggcccattgatccagctcaagctcggctcgatcccgtgcgtcgtggcctcgacgcccgagctcgcgagagagtttcttaagacgaacgagctcgcgttctcctctcgcaagcactctacggccatagacatcgtcacctacgactcgtcctttgctttctctccgtacggaccctactggaagtacatcaagaaactgtgtacctacgagctgctcggagcgaggaacctcggacactttcagcccattaggaatctcgaggtcaggtcctttctgcagcttctgatgcacaagagctttaagggcgagagtgtgaatgtgacagacgagctggtgaggctgacgagcaatgtgatatcccacatgatgctgagcataaggtgctcggaagatgaaggcgatgctgaggcggcgagaacagtgatacgcgaggtgacgcagatatttggggaattcgatgttacggacataatatggttttgcaagaaattcgatctgcaggggataaagaagaggtcagaggatattcagaggaggtatgatgctttgctcgagaagattattagtgatagagagagatcgaggaggcaaaatcgtgataagcatggtggcggtaacaatgaggaggccaaggattttcttgatatgttgcttgatgtgatggagagtggggacacggaggtcaaattcactagagagcatctcaaggctttgattctggatttcttcacggccggtacggacacaacagccatagccaccgagtgggccatcgccgagctcatcaacaacccgaacgtcttgaagaaggcccaagaagaaatatcccggatcatcggaaccaagcggatcgtacaagaatccgacgccccagacctaccctacctccaggccatcatcaaggagacgttccggctccacccaccgatcccgatgctctcgcgtaagtccacctccgattgcacggtcaacggctacaaaatccaagccaagagcctcttgttcgtgaacatatggtccatcggtcgaaaccctaattactgggaaagccctatggagttcaggcccgagcggttcttggagaagggacgcgagtccatcgacgtcaagggccagcactttgagctcttgccttttgggacgggccgcaggggctgtcccggtatgttgctggctatacaagaggtggtcagcatcattgggaccatggttcagtgcttcgactggaaattggcagatggttcgggcaataatgtggacatgaccgaacggtctggattgaccgctccgagagcgttcgatctggtttgccggttgtatccacgggttgacccggccacaatatcgggtgcttgatgtagtagggtgaggcgcgtgttggtgttttatctttcggttttgttctgttagtattattatggtctgtgttgaagcctcaaggatttt

SEQ ID NO: 4 - protein, 512 aa, Torenia hybrida
  Amino acid sequence of flavone synthase
MDTVLITLYTALFVITTTFLLLLRRRGPPSPPGPLSLPIIGHLHLLGPRLHHTFHEFSLKYGPLIQLKLGSIPCVVASTPELAREFLKTNELAFSSRKHSTAIDIVTYDSSFAFSPYGPYWKYIKKLCTYELLGARNLGHFQPIRNLEVRSFLQLLMHKSFKGESVNVTDELVRLTSNVISHMMLSIRCSEDEGDAEAARTVIREVTQIFGEFDVTDIIWFCKKFDLQGIKKRSEDIQRRYDALLEKIISDRERSRRQNRDKHGGGNNEEAKDFLDMLLDVMESGDTEVKFTREHLKALILDFFTAGTDTTAIATEWAIAELINNPNVLKKAQEEISRIIGTKRIVQESDAPDLPYLQAIIKETFRLHPPIPMLSRKSTSDCTVNGYKIQAKSLLFVNIWSIGRNPNYWESPMEFRPERFLEKGRESIDVKGQHFELLPFGTGRRGCPGMLLAIQEVVSIIGTMVQCFDWKLADGSGNNVDMTERSGLTAPRAFDLVCRLYPRVDPATISGA

SEQ ID NO: 5 - DNA, 1770 bp, Perilla frutescens
  CDS (21)..(1538): Nucleotide sequence encoding flavone synthase
tgtcgacggagcaagtggaaatggcactgtacgccgccctcttcctcctgtccgccgccgtggtccgctccgttctggatcgaaaacgcgggcggccgccctaccctcccgggccgttccctcttcccatcatcggccacttacacctcctcgggccgagactccaccaaaccttccacgatctgtcccaacggtacgggcccttaatgcagctccgcctcgggtccatccgctgcgtcattgctgcctcgccggagctcgccaaggaatgcctcaagacacacgagctcgtcttctcctcccgcaaacactccaccgccattgatatcgtcacctacgattcatccttcgctttctctccctacgggccttactggaaattcatcaagaaattatgcacctacgagctgctcggggcccgaaatctcgcccactttcagcccatcaggactctcgaagtcaagtctttcctccaaattcttatgcgcaagggtgaatcgggggagagcttcaacgtgactgaggagctcgtgaagctgacgagcaacgtcatatcgcatatgatgctgagcatacggtgttcagagacggagtcggaggcggaggcggcgaggacggtgattcgggaggtcacgcagatatttggggagttcgacgtctccgacatcatatggctttgtaagaacttcgatttccaaggtataaggaagcggtccgaggatatccagaggagatatgatgctctgctggagaagatcatcaccgacagagagaagcagaggcggacccacggcggcggtggcggcggcggggaagccaaggattttcttgacatgttcctcgacataatggagagcgggaaagccgaagttaaattcacgagggagcatctcaaagctttgattctggatttcttcaccgccggcaccgacacgacggcgatcgtgtgtgaatgggcgatagcagaagtgatcaacaatccaaatgtgttgaagaaagctcaagaagagattgccaacatcgtcggattcgacagaattctgcaagaatccgacgccccaaatctgccctaccttcaagccctcatcaaagaaacattccggctccaccctccaatcccaatgctggcgaggaaatcgatctccgactgcgtcatcgacggctacatgattccggccaacacgctgctcttcgtcaacctctggtccatggggcggaaccctaaaatctgggactacccgacggcgttccagccggagaggtttctggagaaggaaaaggccgccatcgatgttaaagggcagcattttgagctgctaccgttcggaacgggcaggagaggctgcccagggatgcttttagccattcaggaggtggtcatcataattgggacgatgattcaatgcttcgattggaagctgcccgacggctccggccatgttgatatggcagaacggccagggctcacggcaccgcgagagaccgatttgttttgccgtgtggtgccgcgagttgatccgttggttgtttccacccagtgatcaccccctttaaatttattaatgatatatttttattttgagaaaaaataaaaatgctaattgttttgtttcatgatgtaattgttaattagtttctattgtgcgctgtcgcgtgtcgcgtggcttaagataagattgtatcattggtacctaggatgtattttcattttcaataaattattttgtgctgtgtatattaaaaaaaaaaaagaaaaaaaaaaaaaaaaaa

SEQ ID NO: 6 - protein, 506 aa, Perilla frutescens
  Amino acid sequence of flavone synthase
MALYAALFLLSAAVVRSVLDRKRGRPPYPPGPFPLPIIGHLHLLGPRLHQTFHDLSQRYGPLMQLRLGSIRCVIAASPELAKECLKTHELVFSSRKHSTAIDIVTYDSSFAFSPYGPYWKFIKKLCTYELLGARNLAHFQPIRTLEVKSFLQILMRKGESGESFNVTEELVKLTSNVISHMMLSIRCSETESEAEAARTVIREVTQIFGEFDVSDIIWLCKNFDFQGIRKRSEDIQRRYDALLEKIITDREKQRRTHGGGGGGGEAKDFLDMFLDIMESGKAEVKFTREHLKALILDFFTAGTDTTAIVCEWAIAEVINNPNVLKKAQEEIANIVGFDRILQESDAPNLPYLQALIKETFRLHPPIPMLARKSISDCVIDGYMIPANTLLFVNLWSMGRNPKIWDYPTAFQPERFLEKEKAAIDVKGQHFELLPFGTGRRGCPGMLLAIQEVVIIIGTMIQCFDWKLPDGSGHVDMAERPGLTAPRETDLFCRVVPRVDPLVVSTQ

SEQ ID NO: 7 - DNA, 1781 bp, Viola x wittrockiana
  CDS (7)..(1524): Nucleotide sequence encoding flavonoid-3′, 5′-hydroxylase
gacaacatggcaattctagtcaccgacttcgttgtcgcggctataattttcttgatcactcggttcttagttcgttctcttttcaagaaaccaacccgaccgctccccccgggtcctctcggttggcccttggtgggcgccctccctctcctaggcgccatgcctcacgtcgcactagccaaactcgctaagaagtatggtccgatcatgcacctaaaaatgggcacgtgcgacatggtggtcgcgtccacccccgagtcggctcgagccttcctcaaaacgctagacctcaacttctccaaccgcccacccaacgcgggcgcatcccacctagcgtacggcgcgcaggacttagtcttcgccaagtacggtccgaggtggaagactttaagaaaattgagcaacctccacatgctaggcgggaaggcgttggatgattgggcaaatgtgagggtcaccgagctaggccacatgcttaaagccatgtgcgaggcgagccggtgcggggagcccgtggtgctggccgagatgctcacgtacgccatggcgaacatgatcggtcaagtgatactcagccggcgcgtgttcgtgaccaaagggaccgagtctaacgagttcaaagacatggtggtcgagttgatgacgtccgccgggtacttcaacatcggtgacttcataccctcgatcgcttggatggatttgcaagggatcgagcgagggatgaagaagctgcacacgaagtttgatgtgttattgacgaagatggtgaaggagcatagagcgacgagtcatgagcgcaaagggaaggcagatttcctcgacgttctcttggaagaatgcgacaatacaaatggggagaagcttagtattaccaatatcaaagctgtccttttgaatctattcacggcgggcacggacacatcttcgagcataatcgaatgggcgttaacggagatgatcaagaatccgacgatcttaaaaaaggcgcaagaggagatggatcgagtcatcggtcgtgatcggaggctgctcgaatcggacatatcgagcctcccgtacctacaagccattgctaaagaaacgtatcgcaaacacccgtcgacgcctctcaacttgccgaggattgcgatccaagcatgtgaagttgatggctactacatccctaaggacgcgaggcttagcgtgaacatttgggcgatcggtcgggacccgaatgtttgggagaatccgttggagttcttgccggaaagattcttgtctgaagagaatgggaagatcaatcccggtgggaatgattttgagctgattccgtttggagccgggaggagaatttgtgcggggacaaggatgggaatggtccttgtaagttatattttgggcactttggtccattcttttgattggaaattaccaaatggtgtcgctgagcttaatatggatgaaagttttgggcttgcattgcaaaaggccgtgccgctctcggccttggtcagcccacggttggcctcaaacgcgtacgcaacctgagctaatgggctgggcctagttttgtgggccttaatttagagacttttgtgttttaaggtgtgtactttattaattgggtgcttaaatgtgtgttttaatttgtatttatggttaattatgactttattgtataattatttatttttcccttctgggtattttatccatttaatttttcttcagaattatgatcatagttatcagaataaaattgaaaataatgaatcggaaaaaaaaaaaaaaaaaaaaaaaaa

SEQ ID NO: 8 - protein, 506 aa, Viola x wittrockiana
  Amino acid sequence of flavonoid-3′,5′ -hydroxylase
MAILVTDFVVAAIIFLITRFLVRSLFKKPTRPLPPGPLGWPLVGALPLLGAMPHVALAKLAKKYGPIMHLKMGTCDMVVASTPESARAFLKTLDLNFSNRPPNAGASHLAYGAQDLVFAKYGPRWKTLRKLSNLHMLGGKALDDWANVRVTELGHMLKAMCEASRCGEPVVLAEMLTYAMANMIGQVILSRRVFVTKGTESNEFKDMVVELMTSAGYFNIGDFIPSIAWMDLQGIERGMKKLHTKFDVLLTKMVKEHRATSHERKGKADFLDVLLEECDNTNGEKLSITNIKAVLLNLFTAGTDTSSSIIEWALTEMIKNPTILKKAQEEMDRVIGRDRRLLESDISSLPYLQAIAKETYRKHPSTPLNLPRIAIQACEVDGYYIPKDARLSVNIWAIGRDPNVWENPLEFLPERFLSEENGKINPGGNDFELIPFGAGRRICAGTRMGMVLVSYILGTLVHSFDWKLPNGVAELNMDESFGLALQKAVPLSALVSPRLASNAYAT

SEQ ID NO: 9 - DNA, 1014 bp, Torenia hybrida
  CDS (69)..(785): Nucleotide sequence encoding methyltransferase
cggcacgagttcaattccgccattttctccaataataacattcataaatacaatcagcagcagcaaaaatgaaagataagttctatggcaccattttgcagagcgaagccctcgcaaagtatctgttagagacaagtgcctatccacgagaacatccgcagctcaaagaactaaggagcgcaactgtggacaagtatcaatattggagcttgatgaatgttccagctgatgaggggcagttcatttcaatgttactgaaaattatgaacgcaaaaaagacaattgaagttggagttttcacaggctactcactcctatcaactgctctggctctacctgatgatggcaaaatcgttgccattgatcctgatagagaagcttatgagactggtttgccatttatcaagaaagcaaacgtggctcataaaatccaatacatacaatctgatgccatgaaagtcatgaatgacctcattgctgccaagggagaagaagaagaggggagctttgactttgggttcgtggatgcagacaaagaaaactacataaactaccacgagaaactgttgaagctggttaaggttggagggatcataggatacgacaacactctgtggtctggaacagttgctgcatctgaagacgatgagaataatatgcaagactacttaagaggttgcagagggcatatcctcaaactaaactcctttctcgcaaacgatgatcggattgaattggctcacctctctattggagatggactcaccttgtgcaaacgtctcaaataataattttcaactttattattattgtttcataaaaagcatttactgctggcctggcctggcctgtttcagcatcttatatttctattgttctaaatattttagttatcttgtttatcaacttgtctgtcttatatgtttaaaagaaagatgtcatgtaattgtaactcgatcgggctcttgtaatattataatgaattttattgatttcaaaaaaaaaaaaaaaaaa

SEQ ID NO: 10 - protein, 239 aa, Torenia hybrida
  Amino acid sequence of methyltransferase
MKDKFYGTILQSEALAKYLLETSAYPREHPQLKELRSATVDKYQYWSLMNVPADEGQFISMLLKIMNAKKTIEVGVFTGYSLLSTALALPDDGKIVAIDPDREAYETGLPFIKKANVAHKIQYIQSDAMKVMNDLIAAKGEEEEGSFDFGFVDADKENYINYHEKLLKLVKVGGIIGYDNTLWSGTVAASEDDENNMQDYLRGCRGHILKLNSFLANDDRIELAHLSIGDGLTLCKRLK